SEQUENCE LISTING (21 sequences)

SEQ ID NO: 1 - protein, 105 aa, artificial
  Self-assembling protein
MEEVVLITVPSESVARIIAKALVASRLAACVNIVPGLTSIYRWQGSVVEDQELLLLVKTTTHAFPKLKHTVKIIHPYTVPEIVALPIAEGNREYLDWLRENTGLE

SEQ ID NO: 2 - protein, 234 aa, artificial
  Self-assembling protein
MHNNRLQLSRLERVYQSEQAEKLLLAGVMLRDPARFDLRGSLTHGRDVEIDTNVIIEGNVSLGNRVKIGTGCVIKNSAIGDDCEISPYTVVEDAVLAAACTIGPFARLRPGAVLLEGAHVGNFVEMKKAVLGKGSKAGHLTYLGDAAIGDNVNIGAGTITCNYDGANKFTTIIGDDVFVGSDTQLVAPVSVGKGATIAAGTTVTRNVGANALAISRVPQTQKEGWRRPVKKKLE

SEQ ID NO: 3 - protein, 101 aa, artificial
  Self-assembling protein
MEAVRAYELQLELQQIRTLRQSLELKAKELEYAAGIITSLKSERRIYRAFSDLLVEITKLEAIEHIARSIIVYVREIAKLAKRETEIMEELSKLRAPLSLE

SEQ ID NO: 4 - protein, 240 aa, artificial
  Self-assembling protein
MGFQGPLGSHMTISPKEKEKIAIHEAGHDLMGLVSDDDDKVHKISIIPRGMALGVTQQLPIEDKHIYDKKDLYNKILVLLGGRAAEEVFFGKDGITTGAENDLQRATDLAYRMVSMWGMSDKVGPIAIRRVANPFLGGMTTAVDTSPDLLREIDEEVKRIITEQYEKAKAIVEEYKLPLKFVVAALLHSETILCSLFAEVFKTFGIELKDKCKKEELFDKDRKSEENKELKSEEVKEEVV

SEQ ID NO: 5 - protein, 213 aa, artificial
  Self-assembling protein
MGFQGPLGSHMTISPKEKEKIAIHEAGHDLMGLVSDDDDKVHKISIIPRGMALGVTQQLPIEDKHIYDKKDLYNKILVLLGGRAAEEVFFGKDGITTGAENDLQRATDLAYRMVSMWGMSDKVGPIAIRRVANPFLGGMTTAVDTSPDLLREIDEEVKRIITEQYEKAKAIVEEYKLPLKFVVAALLHSETILCSLFAEVFKTFGIELKDKCK

SEQ ID NO: 6 - protein, 355 aa, artificial
  Spycatcher protein
MGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIGSGSGGMHNNRLQLSRLERVYQSEQAEKLLLAGVMLRDPARFDLRGSLTHGRDVEIDTNVIIEGNVSLGNRVKIGTGCVIKNSAIGDDCEISPYTVVEDAVLAAACTIGPFARLRPGAVLLEGAHVGNFVEMKKAVLGKGSKAGHLTYLGDAAIGDNVNIGAGTITCNYDGANKFTTIIGDDVFVGSDTQLVAPVSVGKGATIAAGTTVTRNVGANALAISRVPQTQKEGWRRPVKKK

SEQ ID NO: 7 - protein, 342 aa, Homo sapiens
MLTVEVEVKITADDENKAEEIVKRVIDEVEREVQKQYPNATITRTLTRDDGTVELRIKVKADTEEKAKSIIKLIEERIEEELRKRDPNATITRTVRTEVGSSWSGSGSGGMHNNRLQLSRLERVYQSEQAEKLLLAGVMLRDPARFDLRGSLTHGRDVEIDTNVIIEGNVSLGNRVKIGTGCVIKNSAIGDDCEISPYTVVEDAVLAAACTIGPFARLRPGAVLLEGAHVGNFVEMKKAVLGKGSKAGHLTYLGDAAIGDNVNIGAGTITCNYDGANKFTTIIGDDVFVGSDTQLVAPVSVGKGATIAAGTTVTRNVGANALAISRVPQTQKEGWRRPVKKK

SEQ ID NO: 8 - protein, 389 aa, Homo sapiens
MADQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAKGSGSGSGGMHNNRLQLSRLERVYQSEQAEKLLLAGVMLRDPARFDLRGSLTHGRDVEIDTNVIIEGNVSLGNRVKIGTGCVIKNSAIGDDCEISPYTVVEDAVLAAACTIGPFARLRPGAVLLEGAHVGNFVEMKKAVLGKGSKAGHLTYLGDAAIGDNVNIGAGTITCNYDGANKFTTIIGDDVFVGSDTQLVAPVSVGKGATIAAGTTVTRNVGANALAISRVPQTQKEGWRRPVKKK

SEQ ID NO: 9 - protein, 348 aa, Homo sapiens
MGAGTAQEFVNCKIQPGKVVVFIKPTCPYCRRAQEILSQLPIKQGLLEFVDITATNHTNEIQDYLQQLTGARTVPRVFIGKDCIGGCSDLVSLQQSGELLTRLKQIGALQGSGSGGMHNNRLQLSRLERVYQSEQAEKLLLAGVMLRDPARFDLRGSLTHGRDVEIDTNVIIEGNVSLGNRVKIGTGCVIKNSAIGDDCEISPYTVVEDAVLAAACTIGPFARLRPGAVLLEGAHVGNFVEMKKAVLGKGSKAGHLTYLGDAAIGDNVNIGAGTITCNYDGANKFTTIIGDDVFVGSDTQLVAPVSVGKGATIAAGTTVTRNVGANALAISRVPQTQKEGWRRPVKKK

SEQ ID NO: 10 - protein, 340 aa, Homo sapiens
MERVVINISGLRFETQLKTLCQFPETLLGDPKRRMRYFDPLRNEYFFDRNRPSFDAILYYYQSGGRIRRPVNVPIDIFSEEIRFYQLGEEAMEKFREDEGFLGSGSGGMHNNRLQLSRLERVYQSEQAEKLLLAGVMLRDPARFDLRGSLTHGRDVEIDTNVIIEGNVSLGNRVKIGTGCVIKNSAIGDDCEISPYTVVEDAVLAAACTIGPFARLRPGAVLLEGAHVGNFVEMKKAVLGKGSKAGHLTYLGDAAIGDNVNIGAGTITCNYDGANKFTTIIGDDVFVGSDTQLVAPVSVGKGATIAAGTTVTRNVGANALAISRVPQTQKEGWRRPVKKK

SEQ ID NO: 11 - protein, 330 aa, Homo sapiens
MGMLPRLCCLEKGPNGYGFHLHGEKGKLGQYIRLVEPGSPAEKAGLLAGDRLVEVNGENVEKETHQQVVSRIRAALNAVRLLVVDPETSTTLGSGSGGMHNNRLQLSRLERVYQSEQAEKLLLAGVMLRDPARFDLRGSLTHGRDVEIDTNVIIEGNVSLGNRVKIGTGCVIKNSAIGDDCEISPYTVVEDAVLAAACTIGPFARLRPGAVLLEGAHVGNFVEMKKAVLGKGSKAGHLTYLGDAAIGDNVNIGAGTITCNYDGANKFTTIIGDDVFVGSDTQLVAPVSVGKGATIAAGTTVTRNVGANALAISRVPQTQKEGWRRPVKKK

SEQ ID NO: 12 - protein, 339 aa, Homo sapiens
MAEGNTLISVDYEIFGKVQGVFFRKHTQAEGKKLGLVGWVQNTDRGTVQGQLQGPISKVRHMQEWLETRGSPKSHIDKANFNNEKVILKLDYSDFQIVKGSGSGSGGMHNNRLQLSRLERVYQSEQAEKLLLAGVMLRDPARFDLRGSLTHGRDVEIDTNVIIEGNVSLGNRVKIGTGCVIKNSAIGDDCEISPYTVVEDAVLAAACTIGPFARLRPGAVLLEGAHVGNFVEMKKAVLGKGSKAGHLTYLGDAAIGDNVNIGAGTITCNYDGANKFTTIIGDDVFVGSDTQLVAPVSVGKGATIAAGTTVTRNVGANALAISRVPQTQKEGWRRPVKKK

SEQ ID NO: 13 - protein, 371 aa, Homo sapiens
MVDAFLGTWKLVDSKNFDDYMKSLGVGFATRQVASMTKPTTIIEKNGDILTLKTHSTFKNTEISFKLGVEFDETTADDRKVKSIVTLDGGKLVHLQKWDGQETTLVRELIDGKLILTLTHGTAVCTRTYEKEAGSGSGGMHNNRLQLSRLERVYQSEQAEKLLLAGVMLRDPARFDLRGSLTHGRDVEIDTNVIIEGNVSLGNRVKIGTGCVIKNSAIGDDCEISPYTVVEDAVLAAACTIGPFARLRPGAVLLEGAHVGNFVEMKKAVLGKGSKAGHLTYLGDAAIGDNVNIGAGTITCNYDGANKFTTIIGDDVFVGSDTQLVAPVSVGKGATIAAGTTVTRNVGANALAISRVPQTQKEGWRRPVKKK

SEQ ID NO: 14 - protein, 346 aa, Homo sapiens
MNDSEFHRLADQLWLTIEERLDDWDGDSDIDCEINGGVLTITFENGSKIIINRQEPLHQVWLATKQGGYHFDLKGDEWICDRSGETFWDLLEQAATQQAGETVSFRGSGSGSGGMHNNRLQLSRLERVYQSEQAEKLLLAGVMLRDPARFDLRGSLTHGRDVEIDTNVIIEGNVSLGNRVKIGTGCVIKNSAIGDDCEISPYTVVEDAVLAAACTIGPFARLRPGAVLLEGAHVGNFVEMKKAVLGKGSKAGHLTYLGDAAIGDNVNIGAGTITCNYDGANKFTTIIGDDVFVGSDTQLVAPVSVGKGATIAAGTTVTRNVGANALAISRVPQTQKEGWRRPVKKK

SEQ ID NO: 15 - protein, 320 aa, Homo sapiens
MGTPRARPCRVSTADRKVRKGIMAHSLEDLLNKVQDILKLKDKPFSLVLEEDGTIVETEEYFQALAKDTMFMVLLAGAKWKPGSGSGGMHNNRLQLSRLERVYQSEQAEKLLLAGVMLRDPARFDLRGSLTHGRDVEIDTNVIIEGNVSLGNRVKIGTGCVIKNSAIGDDCEISPYTVVEDAVLAAACTIGPFARLRPGAVLLEGAHVGNFVEMKKAVLGKGSKAGHLTYLGDAAIGDNVNIGAGTITCNYDGANKFTTIIGDDVFVGSDTQLVAPVSVGKGATIAAGTTVTRNVGANALAISRVPQTQKEGWRRPVKKK

SEQ ID NO: 16 - protein, 333 aa, Homo sapiens
MGSRSLQLDKLVNEMTQHYENSVPEDLTVHVGDIVAAPLPTNGSWYRARVLGTLENGNLDLYFVDFGDNGDCPLKDLRALRSDFLSLPFQAIECSGSGSGGMHNNRLQLSRLERVYQSEQAEKLLLAGVMLRDPARFDLRGSLTHGRDVEIDTNVIIEGNVSLGNRVKIGTGCVIKNSAIGDDCEISPYTVVEDAVLAAACTIGPFARLRPGAVLLEGAHVGNFVEMKKAVLGKGSKAGHLTYLGDAAIGDNVNIGAGTITCNYDGANKFTTIIGDDVFVGSDTQLVAPVSVGKGATIAAGTTVTRNVGANALAISRVPQTQKEGWRRPVKKK

SEQ ID NO: 17 - protein, 13 aa, artificial
  Spytag protein
AHIVMVDAYKPTK

SEQ ID NO: 18 - protein, 103 aa, artificial
  Self-assembling protein
MEEVVLITVPSEEVARTIAKALVEERLAACVNIVPGLTSIYRWQGEVVEDQELLLLVKTTTHAFPKLKERVKALHPYTVPEIVALPIAEGNREYLDWLRENTG

SEQ ID NO: 19 - protein, 232 aa, artificial
  Self-assembling protein
MHNNRLQLSRLERVYQSEQAEKLLLAGVMLRDPARFDLRGTLTHGRDVEIDTNVIIEGNVTLGHRVKIGTGCVIKNSVIGDDCEISPYTVVEDANLAAACTIGPFARLRPGAELLEGAHVGNFVEMKKARLGKGSKAGHLTYLGDAEIGDNVNIGAGTITCNYDGANKFKTIIGDDVFVGSDTQLVAPVTVGKGATIAAGTTVTRNVGENALAISRVPQTQKEGWRRPVKKK

SEQ ID NO: 20 - protein, 99 aa, artificial
  Self-assembling protein
MEAVRAYELQLELQQIRTLRQSLELKMKELEYAEGIITSLKSERRIYRAFSDLLVEITKDEAIEHIERSRLVYKREIEKLKKREKEIMEELSKLRAPLS

SEQ ID NO: 21 - protein, 238 aa, artificial
  Self-assembling protein
FQGPLGSHMTISPKEKEKIAIHEAGHALMGLVSDDDDKVHKISIIPRGMALGVTQQLPIEDKHIYDKKDLYNKILVLLGGRAAEEVFFGKDGITTGAENDLQRATDLAYRMVSMWGMSDKVGPIAIRRVANPFLGGMTTAVDTSPDLLREIDEEVKRIITEQYEKAKAIVEEYKEPLKAVVKKLLEKETITCEEFVEVFKLYGIELKDKCKKEELFDKDRKSEENKELKSEEVKEEVV

(19) United States
(12) Patent Application Publication
(10) Pub. No.: US 2016/0369264 A1
(43) Pub. Date: Dec. 22, 2016

(54) SELF-ASSEMBLING TWO-DIMENSIONAL PROTEIN ARRAYS

(71) Applicants: Howard Hughes Medical Institute, Chevy Chase, MD (US); University of Washington, Seattle, WA (US)

(72) Inventors: Tamir Gonen, Ashburn, VA (US); Shane Gonen, Chevy Chase, MD (US); Timothee Lionnet, Sterling, VA (US); David Baker, Seattle, WA (US); Frank DiMaio, Seattle, WA (US); Brian English, Arlington, VA (US); Harve Rouault, Ashburn, VA (US)

(21) Appl. No.: 15/187,599

(22) Filed: Jun. 20, 2016

Related U.S. Application Data
(60) Provisional application No. 62/182,368, filed on Jun. 19, 2015.

(51) Int. Cl. C12N 15/10; G06F 19/16; G01N 33/68

(52) U.S. Cl. CPC C12N 15/1037; G01N 33/6803; G06F 19/16; G01N 33/6845; G01N 2610/00

(57) ABSTRACT

This document relates to two dimensional (2D) protein arrays can be used in biotechnology applications, as well as methods of making and using 2D protein arrays. In some cases, a 2D protein array can be used to evaluate (e.g., image) a structure (e.g., a three dimensional (3D) structure) of a protein of interest. In some cases, a 2D protein array can be used to evaluate (e.g., characterize) protein-protein interactions (e.g., stable interactions vs. transient interactions). In some cases, a 2D protein array can be used to evaluate a binding domain in a protein of interest. In some cases, a 2D protein array can be used to evaluate (e.g., identify) binding targets and/or partners of a protein of interest.

### CROSS-REFERENCE TO RELATED APPLICATIONS

This application claims the benefit of U.S. Provisional Application No. 62/182,368, filed Jun. 19, 2015. The disclosure of the prior application is incorporated by reference in its entirety.

### STATEMENT REGARDING FEDERAL FUNDING

This invention was made with government support under grant no. FA9550-12-1-0112, awarded by the Air Force Office of Scientific Research, and under grant no. N00024-10-D-6318/002, awarded by the Defense Threat Reduction Agency. The government has certain rights in the invention.

### SEQUENCE LISTING

The instant application includes a sequence listing in electronic format submitted to the United States Patent and Trademark Office via the electronic filing system. The ASCII text file, which is incorporated-by-reference herein, is titled “30872-0012001_ST25.txt,” was created on Jun. 20, 2016, has a size of 48 kilobytes.

### BACKGROUND

1. Technical Field

This document relates to methods and materials for making and using two dimensional (2D) protein arrays. For example, this document relates to designing 2D protein arrays for use in biotechnology applications. In some cases, a 2D protein array can be used to evaluate (e.g., image) a structure (e.g., a three dimensional (3D) structure) of a protein of interest. In some cases, a 2D protein array can be used to evaluate (e.g., characterize) protein-protein interactions (e.g., stable interactions vs. transient interactions). In some cases, a 2D protein array can be used to evaluate a binding domain in a protein of interest. In some cases, a 2D protein array can be used to evaluate (e.g., identify) a binding target and/or a binding partner of a protein of interest.

2. Background Information

Programmed self-assembly provides a route to patterning matter at the atomic scale. DNA origami methods (Seeman, Annual review of biochemistry 79, 65-87 (2010); Rothemund, Nature 440, 297-302 (2006)) have been used to generate a wide variety of ordered structures, but progress in designing protein assemblies has been slower owing to the greater complexity of protein-protein interactions. Although proteins that form ordered 3D crystals have been designed (Lanci et al., Proc. Nat. Acad. Sci. USA 109, 7304-7309 (2012)) and 2D lattices have been generated by genetically fusing or chemically cross-linking oligomers with appropriate point symmetric groups (Sinclair et al., Nature nanotechnology 6, 558-562 (2011); Zhang et al., Current opinion in structural biology 27, 79-86 (2014); Brodin et al., Nature chemistry 4, 375-382 (2012); Baneyx et al., Current opinion in biotechnology 28, 39-45 (2014)), there has been little success in designing self-assembling 2D lattices with order sufficient to diffract electrons or x-rays below 15 Å resolution (Sinclair et al., Nature nanotechnology 6, 558-562 (2011)).

### SUMMARY

This document provides methods and materials for making and using 2D protein arrays. For example, a 2D protein array provided herein can include a plurality of oligomeric protein unit cells (e.g., multimeric substructures) having self-assembling proteins and having at least one axis of rotational symmetry. Such 2D protein arrays can be used in biotechnology applications. In some cases, a 2D protein array can be used to evaluate (e.g., image) a structure (e.g., a 3D structure) of a protein of interest. In some cases, a 2D protein array can be used to evaluate (e.g., characterize) protein-protein interactions (e.g., stable interactions vs. transient interactions). In some cases, a 2D protein array can be used to evaluate a binding domain in a protein of interest. In some cases, a 2D protein array can be used to evaluate (e.g., identify) a binding target and/or a binding partner of a protein of interest.

As described herein, protein homo-oligomers can be placed into a 2D layer group and used to form 2D protein arrays mediated by noncovalent protein-protein interfaces. The 2D protein array described herein provides new avenues for processes requiring a 2D array of proteins never before afforded by traditional methods of crystallography, design or fusions. The ease of use afforded by these methods and materials allows for the crystal structure of any small monomeric protein to be obtained in a matter of days, where the main time input is the production of DNA and the expression of protein in the Escherichia coli expression system. The 2D protein array described herein allows for high-throughput testing of thousands of proteins of interest with a high success rate for crystal formation with minimal cost. The flexibility of the method is also important, allowing assembly both intracellularly (e.g., within a living cell) and extracellularly (e.g., in vitro) in order to fit a myriad of environmental conditions.

In some aspects, this document provides 2D protein arrays that contain a plurality of oligomeric protein unit cells, where each oligomeric protein unit cell has at least one axis of rotational symmetry and contains a plurality of self-assembling proteins. The plurality of oligomeric protein unit cells interact with one another at one or more symmetrically repeated protein-protein interfaces to form a 2D protein array. The interaction between the oligomeric protein unit cells can be a non-covalent interaction. The axis of rotational symmetry can be cyclic or dihedral. The one or more symmetrically repeated protein-protein interfaces can include two, three, or four symmetrically repeated protein-protein interfaces. The oligomeric protein unit cell can be a dimeric protein unit cell, a trimeric protein unit cell, a tetrameric protein unit cell, a pentameric protein unit cell, or a hexameric protein unit cell. The at least one axis of rotational symmetry can be the z axis. The oligomeric protein unit cell can have a surface area of greater than 400 Å². The oligomeric protein unit cell can have a shape complementarity of about 0.1 Sc to about 10 Sc (e.g., about 0.5 Sc to about 1.8 Sc). The plurality of self-assembling proteins includes a self-assembling protein which can be p3Z_11 (SEQ ID NO: 1); p3Z_42 (SEQ ID NO: 2); p4Z_9 (SEQ ID NO: 3); p6_9H (SEQ ID NO: 4); or p6_9H_KDKCKXX (SEQ ID NO: 5). The plurality of self-assembling proteins includes a self-assembling protein that can be about 25 to about 500 amino acids in length (e.g., about 200 to about 250 amino acids in length). At least one of the plurality of self-assembling proteins can be a self-assembling fusion protein. The self-assembling fusion protein can include a self-assembling protein fused to a protein of interest. The self-assembling fusion protein can also include a linker between the self-assembling protein and the protein of interest. The linker can include a glycine-glycine or a glycine-serine. The protein of interest can be a protein with an unknown 3D structure. The protein of interest can be a protein with an unknown binding partner. The 2D protein array can have a thickness of about 0.1 nM to about 100 nM (e.g., about 3 nM to about 8 nM). The 2D protein array can have a length of about 0.05 μm to about 5 (e.g., about 1 μm).

In some aspects, this document provides a method of assembling a 2D protein array. Such methods can include, or consist essentially of, providing a plurality of self-assembling proteins under conditions that allow the self-assembling proteins to interact with one another to form a plurality of oligomeric protein unit cells, where each oligomeric protein unit cell contains at least one axis of rotational symmetry, and where the plurality of oligomeric protein unit cells interact with each other at one or more symmetrically repeated protein-protein interfaces to form the 2D protein array. Providing a plurality of self-assembling proteins can include expressing said plurality of self-assembling proteins from a cell-based expression system. The cell-based expression system can be a bacterial expression system (e.g., an Escherichia coli expression system). The 2D protein array can be formed intracellularly.

In some aspects, this document provides a method for determining a 3D structure of a protein of interest. Such methods can include, or consist essentially of, providing a plurality of self-assembling fusion proteins containing a self-assembling fusion protein fused to the protein of interest under conditions that allow the self-assembling fusion proteins to interact with one another to form a plurality of oligomeric protein unit cells, wherein each of said plurality of oligomeric protein unit cells comprises at least one axis of rotational symmetry, where the plurality of oligomeric protein unit cells interact with each other at one or more symmetrically repeated protein-protein interfaces to form a 2D protein array that presents the protein of interest on its surface, and determining the 3D structure of the protein of interest present on the surface of the 2D protein array. Determining the 3D structure of the protein of interest present on the surface of the 2D protein array can include X-ray crystallography, NMR spectroscopy, or dual polarisation interferometry.

In some aspects, this document provides a method for determining a binding partner of a protein of interest. Such methods can include, or consist essentially of, providing a plurality of self-assembling fusion proteins containing a self-assembling protein fused to the protein of interest under conditions that allow the self-assembling fusion proteins to interact with each other to form a plurality of oligomeric protein unit cells, where each oligomeric protein unit cell contains at least one axis of rotational symmetry, where the plurality of oligomeric protein unit cells interact with each other at one or more symmetrically repeated protein-protein interfaces to form said 2D protein array, where the 2D protein array presents the protein of interest on its surface; providing at least one potential binding target; and determining if the at least one potential binding target is a binding partner of the protein of interest present on the surface of the 2D protein array. Determining if the at least one potential binding target is a binding partner of the protein of interest present on the surface of the 2D protein array can include fluorescence resonance energy transfer. The protein of interest can be labeled with a first detectable label (e.g., a first fluorescent label), and the at least one potential binding target can be labeled with a second detectable label (e.g., a second fluorescent label).

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a computational design strategy and experimental analysis of designed arrays. (A) The P 3 2 1 unit cell with three-fold axes represented by triangles. Yellow (−) and purple (+) C3 objects have opposite orientations along the z axis. Inset indicates the three degrees of freedom of the lattice. (B) p3Z_42 2 D array. (C) p3Z_42 designed interface with “zipper-like” hydrophobic packing and peripheral hydrogen bonds. (D) Large (>1 μm) E. coli grown array (middle), higher magnification view with lattice spacing as in (b) (right), and Fourier transform (amplitudes) of the large array (left). (E) Left: 15 Å projection map calculated from a large array. Right: overlay of the p3Z_42 design model on the projection map. (F) The P 4 21 2 lattice. Ovals represent two-fold axes and squares, four-fold axes. (G) p4Z_9 array. (H) p4Z_9 designed interface. (I) Negatively stained E. coli grown array (main panel), an in vitro re-folded lattice at higher magnification (inset), and Fourier transform of the main panel (left). (J) 14 Å projection map calculated from an E. coli array as in (i) without (left) and with (right) p4Z_9 design model. (K) The P 6 lattice has two degrees of freedom (A, θ) (inset) available for sampling. Six-folds are represented by hexagons (L) p6_9H array. (M) p6_9H designed interface. (N) p6_9H lattice grown in vivo with Fourier transform at left and higher magnification view at right. (O) 14 Å projection map of p6_9H from E. coli grown arrays as in (n) and cartoon overlay (right). All scale bars: Black=5 nm, White=50 nm.

FIG. 2 shows cryo-EM analysis of design p3Z_42. (A) Cryo-EM micrograph of E. coli grown p3Z_42 recorded from non-purified, re-suspended insoluble material. (B) Fourier transform calculated from motion-corrected movies taken from samples like in (a). (C) Electron-diffraction of a crystal as in (a) (D) 4 Å projection map calculated from motion-corrected movies from material as in (a) showing a linked repeat protein arrangement similar to the p3Z_42 design model. The unit cell is shown in blue and contains two alternating trimeric units. Triangular density at the corners of the unit cell is likely an averaging artifact. (E) p3Z_42 design model in a similar view as in (d). Scale bar=50 nm.

FIG. 3 shows design p3Z_11 in P 3 2 1 symmetry. (A) Design p3Z_11 shown in VDW space filled view with the purple and yellow proteins oriented 180° from each other on Z axis in P 3 2 1 symmetry, similar to p3Z_42 design. (B) In-plane view of the p3Z_11 design showing the change in z height between the trimeric subunits. Lattice thickness by design=˜4 nm (C) p3Z_11 design interface showing a large hydrophobic patch made of six isoleucines flanked by hydrogen bond networks. Transparent VDW interface area is also shown to highlight the lock-and-key docked design between trimeric subunits. (D) Negative-stain micrograph of p3Z_11 showing a large stacking of proteins in 2D to form 3D crystals. The edges of which contain an observable lattice giving spots on a Fourier transform (top right). Scale bars: Black=5 nm, white=50 nm.

FIG. 4 shows in-plane views of p3Z_42, p4Z_9 and p6_9H. (A) p3Z_42 design in-plane view showing a slight difference in z height between neighboring trimers. Lattice design thickness=˜7 nm (B) p4Z_9 design in-plane view highlighting a great difference in z height between neighboring tetrameric proteins. Lattice design thickness=˜8 nm (C) p6_9H design in-plane view showing no difference in z height between neighboring hexameric proteins due to the lack of a z degree of freedom in P 6 symmetry. Lattice design thickness=˜3 nm.

FIG. 5 shows SDS-PAGE gel of (from left to right) p3Z_42, p4Z_9, p6_9H and p3Z_11 protein expression. SN=soluble supernatant, P=insoluble pellet. Expression of p3Z_42, p4Z_9 and p3Z_11 protein is almost exclusively contained in the insoluble pellet material while design p6_9H proteins express mostly in the pellet while some proteins remain soluble.

FIG. 6 shows in vitro array formation of p3Z_42, p4Z_9 and p6_9H designs. (A) Design p3Z_42 expressed using an in vitro expression kit. This negative-stain micrograph was made 4 hours after adding pure plasmid DNA of p3Z_42 to the kit components without purification. A Fourier transform is shown from a crystal in the micrograph showing the same P 3 2 1 lattice as visualized in p3Z_42 E. coli expression. (B) Fast dilution re-folded p4Z_9 protein. Large arrays form analogous to those seen from E. coli expressed protein. A Fourier transform is shown highlighting the square lattice. (C) Dialysis re-folded p4Z_9 protein. Large fibrous structures form with the same square array pattern as in E. coli expressed proteins. Fourier transform is shown highlighting the square repeat pattern. (D) Purified and concentrated protein from p6_9H soluble fractions. Arrays were not visualized at this point. Fourier transform of the image reveals no P 6 repeat pattern. (E) p6_9H array formation from material as in (d). These arrays formed after further concentration of protein as in (d) and heat application in a water bath. The EM grid was prepared by a 50-fold dilution of the concentrated array product, suggesting that once formed, the arrays are very stable in solution. Fourier transform is shown with the same P 6 arrangement seen in the pellet sample. Scale bars=50 nm.

FIG. 7 shows mutagenesis of p6_9 (precursor to p6_9H). (A). Micrograph of negatively-stained p6_9 pellet. Small patches of single-layer, 2D hexamers could be clearly observed. (B) p6_9 protein design highlighting the repeat interface area (blue). (C) Zoom-in view of the p6_9 interface showing E188. (D) Zoom-in view of the p6_9H interface highlighting the E188H mutation made to stabilize the design by forming a hydrogen bond network with neighboring serines on both the same hexamer and the P 6 related hexamer. (E) Micrograph of negatively-stained p6_9H pellet. Larger, more stable 2D arrays could be readily observed in sharp contrast to p6_9. Scale bars=50 nm.

FIG. 8 shows 2D self-crystallization by genetic fusion method overview using GFP as the fusion example (A). Outline of the original designed array, p3Z-42. A C3 symmetric protein was used and the interface between same, inverted monomers (yellow and purple) were designed to noncovalently self-assemble into a p321 lattice. Unit cell is shown in black (B). Using the N-terminus as an example here, the fusion protein (orange) of choice (in this example, GFP) is genetically fused using a short linker (red), usually a GS or GG motif, to the original p3Z-42 protein monomer. This protein in turn naturally assembles into a trimer (with three copies of the fusion protein) that then self-assembles into a 2D array as described in (A).

FIG. 9 shows an overview of fusion arrays created. (A) Calmodulin is highlighted whereby very large crystals were seen under negative-stain EM, some reaching 1 um in diameter. A zoom in of the lattice is shown and the resulting FFT with repeat spots to high order even in low-resolution negative-stain. A cartoon representation of the calmodulin protein is shown. (B-F) Cartoon representations and representative negatively stained micrographs of different fusion proteins, integrin binding protein, ferrodoxin, human glutaredoxin, TDRD2 and spycatcher protein respectively.

FIG. 10 shows a 2D p3Z-42-Spycatcher fusion array and detection of Spycatcher-Spytag binding. (A) A 2D p3Z-42-Spycatcher array (the P 6 unit cell is shown in black) was contacted with Spytag labeled with Alexa Fluor® 488 and/or Alexa Fluor® 647. (B) Fluorescent emissions from Alexa Fluor® 488, Alexa Fluor® 647, and combinations thereof at varying ratios (middle panel) demonstrate that binding between Spycatcher (when presented on a 2D p3Z-42-Spycatcher array) and Spytag labeled with Alexa Fluor® 488 and/or Alexa Fluor® 647 can be detected (top panel). The emission intensity for each label (identified by red or green channel) illustrates the proportional increases, showing the consistent transfer of energy in the labeled protein array (bottom panel).

### DETAILED DESCRIPTION

This document provides methods and materials for making and using 2D protein arrays. For example, a 2D protein array provided herein can include a plurality of oligomeric protein unit cells made up of self-assembling proteins and having at least one axis of rotational symmetry. Such 2D protein arrays can be used in biotechnology applications. In some cases, a 2D protein array can be used to evaluate (e.g., image) a structure (e.g., a 3D structure) of a protein of interest. In some cases, a 2D protein array can be used to evaluate (e.g., characterize) protein-protein interactions (e.g., stable interactions vs. transient interactions). In some cases, a 2D protein array can be used to evaluate a binding domain in a protein of interest. In some cases, a 2D protein array can be used to evaluate (e.g., identify) binding targets and/or partners of a protein of interest.

### 2D Protein Array

This document provides 2D protein arrays including a plurality of self-assembling proteins that self-interact to form an oligomeric protein unit cell (also referred to herein as a multimeric substructure) having at least one axis of rotational symmetry. As used herein, a 2D protein array is an ordered protein nanostructure, the assembly of which is mediated by designed protein-protein interfaces stabilized by extensive noncovalent interactions. A 2D protein array may also be referred to herein as a 2D protein nanostructure or a 2D protein ultrastructure. Characteristics of a 2D protein array provided herein can be evaluated using any suitable method.

An oligomeric protein unit cell having at least one axis of rotational symmetry can include a plurality of self-assembling proteins. As used herein, a “plurality” means at least two (e.g., 3, 4, 5, 6, or more) proteins can be included in an oligomeric protein unit cell. In some cases, an oligomeric protein unit cell can be a dimeric protein unit cell (e.g., with two copies of the self-assembling protein), a trimeric protein unit cell (e.g., with three copies of the self-assembling protein), a tetrameric protein unit cell (e.g., with four copies of the self-assembling protein), a pentameric protein unit cell (e.g., with five copies of the self-assembling protein), a or hexameric protein unit cell (e.g., with six copies of the self-assembling protein). An oligomeric protein unit cell described herein can include a plurality of the same self-assembling protein (also referred to as a homo-oligomeric protein unit cell) or a plurality of a two or more different self-assembling proteins (also referred to as a hetero-oligomeric protein unit cell).

Self-assembling proteins within an oligomeric protein unit cell can interact via any appropriate protein-protein interface to form the oligomeric protein unit cell. The protein-protein interface can be a non-covalent protein-protein interaction. Non-covalent interactions include, for example, electrostatic interactions, π-effects, van der Waals forces, hydrogen bonding, and hydrophobic effects. In some cases, the protein-protein interaction can be a synthetic interaction (e.g., designed to self-interact) or a naturally occurring interaction.

An oligomeric protein unit cell described herein can have any appropriate unit cell size. In some cases, an oligomeric protein unit cell can have a size of about 5 to about 12 nm (e.g., about 5 to about 12 nm, about 5 to about 12 nm, about 5 to about 12 nm, or about 5 to about 12 nm). For example, a 2D protein array described herein can include a plurality of oligomeric protein unit cells having an oligomeric protein unit cell size of about 8.5 nm.

An oligomeric protein unit cell having at least one axis of rotational symmetry can have any appropriate rotational symmetry. As used herein, “at least one axis of rotational symmetry” means at least one axis of symmetry around which the oligomeric protein unit cell can be rotated without changing its appearance. The axis around the rotation occurs can be the x, y, z, r, theta (θ), or phi (φ) axis. Examples of oligomeric protein states having symmetry include cyclic, dihedral, cubic, and helical. In some cases, an oligomeric protein unit cell can have cyclic symmetry (e.g., rotation about a single axis). Generally, a, oligomeric protein unit cell with n subunits and cyclic symmetry will have n-fold rotational symmetry, sometimes denoted as Cn symmetry. For example, an oligomeric protein unit cell including trimeric self-assembled proteins can have a three-fold axis. In some cases, an oligomeric protein unit cell can have symmetries with multiple rotational symmetry axes. Examples of symmetries with multiple rotational symmetry axes include dihedral symmetry (e.g., cyclic symmetry plus an orthogonal two-fold rotational axis), and cubic point group symmetry (e.g., tetrahedral, octahedral, and icosahedral point group symmetry).

An oligomeric protein unit cell described herein can have any appropriate 2D layer group. There are seventeen distinct ways (layer groups) in which three-dimensional objects can come together to form periodic two-dimensional layers. Such layer groups are described elsewhere (see, e.g., Nannenga et al., “Overview of electron crystallography of membrane proteins: crystallization and screening strategies using negative stain electron microscopy.” Coligan et al. (Eds.) Current Protocols in Protein Science Chapter 17, Unit 17 15 (2013)). Examples of 2D layer groups include C 2 1 1, P 2 21 21, P 3, P 3 2 1, P 4, P 4 21 2, P 6, C 2 2 2, P 3 1 2, P 4 2 2, and P 6 2 2. In some cases, an oligomeric protein unit cell can have a 2D group layer of P 3 2 1, P 4 21 2, or P 6. For example, a 2D protein array described herein can include a plurality of oligomeric protein unit cells having a 2D group layer of P 3 2 1.

An oligomeric protein unit cell described herein can have any appropriate surface area. In some cases, an oligomeric protein unit cell can have a surface area of about 250 Å² to about 2000 Å² (e.g., about 275 Å² to about 1500 Å², about 300 Å² to about 1250 Å², about 325 Å² to about 1500 Å², or about 350 Å² to about 1000 Å²). In some cases, an oligomeric protein unit cell can have a surface area of greater than 400 Å² (e.g., 425 Å², 450 Å², 475 Å², 500 Å², 525 Å², 552 Å², 575 Å², or 600 Å²).

An oligomeric protein unit cell described herein can have any appropriate shape complementarity. An appropriate shape complementarity can include the largest possible number of contacting amino acids within the self-assembling protein. An appropriate shape complementarity can include the fewest possible number of clashes between contacting amino acids within the self-assembling protein. In some cases, an oligomeric protein unit cell can have a shape complementarity of about 0.1 S_{c} to about 10 S_{c} (e.g., about 0.2 S_{c} to about 9 S_{c}, about 0.3 S_{c} to about 8 S_{c}, about 0.3 S_{c} to about 5 S_{c}, about 0.4 S_{c} to about 2.5 S_{c} or about 0.5 S_{c} to about 1.8 S_{c}). In some cases, an oligomeric protein unit cell can have a shape complementarity of greater than 0.5 S_{c} (e.g., 1 S_{c}, 1.5 S_{c}, 2 S_{c}, 2.5 S_{c}, 3 S_{c}, 3.5 S_{c}, or 4 Sc). For example, at least 50% (e.g., at least 55%, at least 60%, at least 65%, at least 70%, or at least 75%) of the atomic contacts (e.g., amino acids) comprising each symmetrically repeated, non-natural, non-covalent protein-protein interface between proteins of the present invention are formed from amino acid residues residing in elements of alpha helix and/or beta strand secondary structure.

A plurality of oligomeric protein unit cells can interact with each other at one or more (e.g., two, three, four, five, or six) symmetrically repeated protein-protein interfaces to form a 2D protein array. A plurality of oligomeric protein unit cells can include multiple copies of a single unit cell or multiple copies of two or more (e.g., three, four, or five) different oligomeric protein unit cells. Oligomeric protein unit cells provided herein can interact via any appropriate protein-protein interface to form a 2D protein array described herein. The protein-protein interface can be a non-covalent protein-protein interaction. Non-covalent interactions include, for example, electrostatic interactions, π-effects, van der Waals forces, hydrogen bonding, and hydrophobic effects. Oligomeric protein unit cells provided herein can interact at multiple interfaces between the oligomeric protein unit cells. The interfaces between oligomeric protein unit cells can be continuous or discontinuous.

A 2D protein array described herein can be any appropriate size. Generally, a nanostructure (e.g., a 2D protein array) can have at least one dimension on the nanoscale, i.e., between 0.1 and 100 nm. In some cases, a 2D protein array can have a thickness of about 0.1 nm to about 100 nm (e.g., about 0.5 nm to about 75 nm, about 1 nm to about 50 nm, about 1.25 nm to about 25 nm, about 1.5 nm to about 20 nm, about 1.7 nm to about 15 nm, about 2 nm to about 12 nm, or about 2.5 nm to about 10 nm). For example, a 2D protein array can have a thickness of about 3 nm to about 8 nm. In some cases, a 2D protein array can have a length and/or width of about 0.05 micron (μm) to about 5 μm (e.g., about 0.1 μm to about 4 μm, about 0.2 μm to about 3 μm, about 0.3 μm to about 2 μm, about 0.4 μm to about 2.5 μm, about 0.5 μm to about 2 μm, or about 0.8 μm to about 1.5 μm). For example, a 2D protein array can have a length and/or width of about 1 μm. In some cases, a 2D protein array can have a thickness of about 3 nM to about 8 nM and a length of about 1 μm.

A 2D protein array described herein can be attached to a solid support. A 2D protein array described herein can be formed on a solid support. Examples of solid supports include silicon (e.g., silicon chips), glass (e.g., microscope slides), membranes (e.g., nitrocellulose film), polymers (e.g., culture plates such as microtitre plates), beads, resins, and combinations thereof.

In some cases, a 2D protein array provided herein can include a plurality of self-assembling proteins (e.g., p3Z_42) that self-interact to form a trimeric protein unit cell having cyclic rotational symmetry around its axis θ.

### Self-Assembling Proteins

This document provides self-assembling proteins that can form oligomeric protein unit cells which in turn form 2D protein arrays described herein. A self-assembling protein can be from any appropriate source. A self-assembling protein can be synthetic protein or a naturally-occurring protein. For example, a self-assembling protein can be a bacterial, fungal, plant, or mammalian (e.g., human), or a designed protein. A self-assembling protein can be produced by any suitable means, including recombinant production or chemical synthesis.

A self-assembling protein described herein can be any appropriate length. In some cases, a self-assembling protein can be about 25 to about 500 amino acids in length (e.g., about 30 to about 475, about 40 to about 450, about 50 to about 425, about 75 to about 400, about 100 to about 375, about 125 to about 350, about 150 to about 325, or about 175 to about 300). For example, a self-assembling protein can be about 200 to about 250 amino acids in length.

A self-assembling protein described herein can have any appropriate molecular weight. In some cases, a self-assembling protein can have a molecular weight of about 9 kDa to about 35 kDa (e.g., about 10 kDa to about 32 kDa, about 11 kDa to about 30 kDa, about 12 kDa to about 27 kDa, about 13 kDa to about 25 kDa, or about 15 kDa to about 20 kDa). In some cases, a self-assembling protein can be a monomeric protein having a molecular weight less than 17 kDa (e.g., 16 kDa, 15 kDa, 14 kDa, 13 kDa, 12 kDa, 11 kDa, 10 kDa, or 9 kDa).

In some cases, the protein-protein interaction can be a synthetic interaction. For example, the self-assembling protein can be a fully synthetic protein or a variation/derivative of a naturally occurring protein designed to self-interact (e.g., p3Z_11, p3Z_42, p4Z_9, p6_9H, and p6_9H_KDKCKXX). In some cases, the protein-protein interaction can be a naturally occurring interaction. For example, the self-assembling protein can be a naturally occurring protein with an ability to self-interact (e.g., pepsin, alcohol dehydrogenase, porin, neuroamidase, complement C1, phosphofructokinase, aspartate carbanoyltransferase, glycoate oxidase, glutamine synthetase, and ferritin). Exemplary self-assembling proteins can be seen in Table 1.

TABLE 1
Self-assembling proteins.

| | amino acid sequence | SEQ ID NO: |
|---|---|---|
| p3Z_11 | MEEVVLITVPSESVARIIAKALVASRLAACVNIVPGLTSIYRWQGSVVED | 1 |
| | QELLLLVKTTTHAFPKLKHTVKIIHPYTVPEIVALPIAEGNREYLDWLRE | |
| | NTGLE | |
| p3Z_42 | MHNNRLQLSRLERVYQSEQAEKLLLAGVMLRDPARFDLRGSLTHGRD | 2 |
| | VEIDTNVIIEGNVSLGNRVKIGTGCVIKNSAIGDDCEISPYTVVEDAVLA | |
| | AACTIGPFARLRPGAVLLEGAHVGNFVEMKKAVLGKGSKAGHLTYLG | |
| | DAAIGDNVNIGAGTITCNYDGANKFTTIIGDDVFVGSDTQLVAPVSVGK | |
| | GATIAAGTTVTRNVGANALAISRVPQTQKEGWRRPVKKKLE | |
| p4Z_9 | MEAVRAYELQLELQQIRTLRQSLELKAKELEYAAGIITSLKSERRIYRAF | 3 |
| | SDLLVEITKLEAIEHIARSIIVYVREIAKLAKRETEIMEELSKLRAPLSLE | |
| p6_9H | MGFQGPLGSHMTISPKEKEKIAIHEAGHDLMGLVSDDDDKVHKISIIPR | 4 |
| | GMALGVTQQLPIEDKHIYDKKDLYNKILVLLGGRAAEEVFFGKDGITT | |
| | GAENDLQRATDLAYRMVSMWGMSDKVGPIAIRRVANPFLGGMTTAV | |
| | DTSPDLLREIDEEVKRIITEQYEKAKAIVEEYKLPLKFVVAALLHSETILC | |
| | SLFAEVFKTFGIELKDKCKKEELFDKDRKSEENKELKSEEVKEEVV | |
| p6_9H_ | MGFQGPLGSHMTISPKEKEKIAIHEAGHDLMGLVSDDDDKVHKISIIPR | 5 |
| KDKCKXX | GMALGVTQQLPIEDKHIYDKKDLYNKILVLLGGRAAEEVFFGKDGITT | |
| | GAENDLQRATDLAYRMVSMWGMSDKVGPIAIRRVANPFLGGMTTAV | |
| | DTSPDLLREIDEEVKRIITEQYEKAKAIVEEYKLPLKFVVAALLHSETILC | |
| | SLFAEVFKTFGIELKDKCK | |

A self-assembling protein described herein can have at least 75 percent (%) identity (e.g., at least 78% identity, at least 80% identity, at least 82% identity, at least 85% identity, at least 87% identity, at least 89% identity, at least 90% identity, at least 92% identity, at least 95% identity, at least 97% identity, at least 98% identity, or at least 99% identity) to any one of SEQ ID NOs: 1-5 provided the ability to self-interact to form an oligomeric protein unit cell is maintained. In some cases, an amino acid residue within a self-assembling protein that is present on the surface of the formed oligomeric protein unit cell (e.g., residues greater than 5 Å from the protein-protein interface forming the oligomeric protein unit cell and/or residues having a solvent-accessible surface area of greater than 50 Å²) can be substituted with a different amino acid as desired for a given purpose without disruption of protein formation or structure of the oligomeric protein unit cell. In various other embodiments, these same residues can be modified by conservative substitutions. For example, an amino acid residue within a self-assembling protein that is present on the surface of the formed oligomeric protein unit cell can be substituted with a conservative amino acid substitutions.

In some cases, a self-assembling protein (e.g., p3Z_42) can be attached to one or more proteins of interest. A protein of interest can be attached to either N- or C-terminus of a self-assembling protein. Appropriate methods of attaching two proteins (e.g., a self-assembling protein and a protein of interest) include, without limitation, expressing a fusion protein from a nucleic acid sequence encoding both proteins. A 2D protein array including a protein of interest fused to a self-assembling protein can also be referred to as a 2D fusion protein array. In cases where a self-assembling protein is attached to a protein of interest, the 2D protein array can have the protein of interest embedded within the array, the 2D protein array can present the protein of interest on the array surface, or a combination thereof.

A protein of interest can be any appropriate protein such as, for example, enzymes, cell signaling proteins, ligand binding proteins, and structural proteins. In some cases, a protein of interest can have an unknown protein structure. In some cases, a protein of interest can have an unknown binding partner (e.g., a receptor, a ligand, or an analyte). Examples of proteins of interest can be, without limitation, Spycatcher, ferrodoxin, calmodulin, glutaredoxin (e.g., human glutaredoxin), T1 domain of Kv1.3 potassium channel, chemokine receptor (e.g., CXCR2), acylphosphatase (e.g., human acylphosphatase), heart fatty acid binding protein (e.g., human heart fatty acid binding protein), cyaY protein, DFFA-like effector C, and TDRD2. A protein of interest can be full-length protein or a fragment thereof For example, a fragment of a protein of interest can include one or more functional domains such as a binding domain (e.g., zinc finger domain, basic leucine zipper domain, death effector domain (DED), phosphotyrosine-binding domain (PTB), and pleckstrin homology domain (PH)), Src homology 2 domain (SH2), domain of unknown function (DUF), and/or analyte binding domain. A 2D protein array including oligomeric protein unit cells having a protein of interest attached to one or more functional domains can also be referred to as a functionalized 2D protein array. Exemplary proteins of interest can be seen in Table 2.

TABLE 2
Proteins of interest.

| | amino acid sequence | SEQ ID NO: |
|---|---|---|
| Spycatcher | MGAMVDTLSGLSSEQGQSGDMTIEEDSATHIKFSKRDEDGKELAGAT | 6 |
| | MELRDSSGKTISTWISDGQVKDFYLYPGKYTFVETAAPDGYEVATAITF | |
| | TVNEQGQVTVNGKATKGDAHIGSGSGGMHNNRLQLSRLERVYQSEQ | |
| | AEKLLLAGVMLRDPARFDLRGSLTHGRDVEIDTNVIIEGNVSLGNRVK | |
| | IGTGCVIKNSAIGDDCEISPYTVVEDAVLAAACTIGPFARLRPGAVLLEG | |
| | AHVGNFVEMKKAVLGKGSKAGHLTYLGDAAIGDNVNIGAGTITCNY | |
| | DGANKFTTIIGDDVFVGSDTQLVAPVSVGKGATIAAGTTVTRNVGANA | |
| | LAISRVPQTQKEGWRRPVKKK | |
| Ferrodoxin | MLTVEVEVKITADDENKAEEIVKRVIDEVEREVQKQYPNATITRTLTRD | 7 |
| | DGTVELRIKVKADTEEKAKSIIKLIEERIEEELRKRDPNATITRTVRTEV | |
| | GSSWSGSGSGGMHNNRLQLSRLERVYQSEQAEKLLLAGVMLRDPARF | |
| | DLRGSLTHGRDVEIDTNVIIEGNVSLGNRVKIGTGCVIKNSAIGDDCEIS | |
| | PYTVVEDAVLAAACTIGPFARLRPGAVLLEGAHVGNFVEMKKAVLGK | |
| | GSKAGHLTYLGDAAIGDNVNIGAGTITCNYDGANKFTTIIGDDVFVGS | |
| | DTQLVAPVSVGKGATIAAGTTVTRNVGANALAISRVPQTQKEGWRRP | |
| | VKKK | |
| Calmodulin | MADQLTEEQIAEFKEAFSLFDKDGDGTITTKELGTVMRSLGQNPTEAE | 8 |
| | LQDMINEVDADGNGTIDFPEFLTMMARKMKDTDSEEEIREAFRVFDK | |
| | DGNGYISAAELRHVMTNLGEKLTDEEVDEMIREADIDGDGQVNYEEF | |
| | VQMMTAKGSGSGSGGMHNNRLQLSRLERVYQSEQAEKLLLAGVML | |
| | RDPARFDLRGSLTHGRDVEIDTNVIIEGNVSLGNRVKIGTGCVIKNSAI | |
| | GDDCEISPYTVVEDAVLAAACTIGPFARLRPGAVLLEGAHVGNFVEMK | |
| | KAVLGKGSKAGHLTYLGDAAIGDNVNIGAGTITCNYDGANKFTTIIGD | |
| | DVFVGSDTQLVAPVSVGKGATIAAGTTVTRNVGANALAISRVPQTQKE | |
| | GWRRPVKKK | |
| Human | MGAGTAQEFVNCKIQPGKVVVFIKPTCPYCRRAQEILSQLPIKQGLLEF | 9 |
| Glutaredoxin | VDITATNHTNEIQDYLQQLTGARTVPRVFIGKDCIGGCSDLVSLQQSGE | |
| | LLTRLKQIGALQGSGSGGMHNNRLQLSRLERVYQSEQAEKLLLAGVM | |
| | LRDPARFDLRGSLTHGRDVEIDTNVIIEGNVSLGNRVKIGTGCVIKNSAI | |
| | GDDCEISPYTVVEDAVLAAACTIGPFARLRPGAVLLEGAHVGNFVEMK | |
| | KAVLGKGSKAGHLTYLGDAAIGDNVNIGAGTITCNYDGANKFTTIIGD | |
| | DVFVGSDTQLVAPVSVGKGATIAAGTTVTRNVGANALAISRVPQTQKE | |
| | GWRRPVKKK | |
| T1 domain of | MERVVINISGLRFETQLKTLCQFPETLLGDPKRRMRYFDPLRNEYFFDR | 10 |
| Kv1.3 | NRPSFDAILYYYQSGGRIRRPVNVPIDIFSEEIRFYQLGEEAMEKFREDE | |
| Potassium | GFLGSGSGGMHNNRLQLSRLERVYQSEQAEKLLLAGVMLRDPARFDL | |
| Channel | RGSLTHGRDVEIDTNVIIEGNVSLGNRVKIGTGCVIKNSAIGDDCEISPY | |
| | TVVEDAVLAAACTIGPFARLRPGAVLLEGAHVGNFVEMKKAVLGKGS | |
| | KAGHLTYLGDAAIGDNVNIGAGTITCNYDGANKFTTIIGDDVFVGSDT | |
| | QLVAPVSVGKGATIAAGTTVTRNVGANALAISRVPQTQKEGWRRPVK | |
| | KK | |
| Chemokine | MGMLPRLCCLEKGPNGYGFHLHGEKGKLGQYIRLVEPGSPAEKAGLL | 11 |
| Receptor | AGDRLVEVNGENVEKETHQQVVSRIRAALNAVRLLVVDPETSTTLGS | |
| CXCR2 | GSGGMHNNRLQLSRLERVYQSEQAEKLLLAGVMLRDPARFDLRGSLT | |
| | HGRDVEIDTNVIIEGNVSLGNRVKIGTGCVIKNSAIGDDCEISPYTVVE | |
| | DAVLAAACTIGPFARLRPGAVLLEGAHVGNFVEMKKAVLGKGSKAGH | |
| | LTYLGDAAIGDNVNIGAGTITCNYDGANKFTTIIGDDVFVGSDTQLVAP | |
| | VSVGKGATIAAGTTVTRNVGANALAISRVPQTQKEGWRRPVKKK | |
| Human | MAEGNTLISVDYEIFGKVQGVFFRKHTQAEGKKLGLVGWVQNTDRG | 12 |
| Acylphosphatase | TVQGQLQGPISKVRHMQEWLETRGSPKSHIDKANFNNEKVILKLDYS | |
| | DFQIVKGSGSGSGGMHNNRLQLSRLERVYQSEQAEKLLLAGVMLRDP | |
| | ARFDLRGSLTHGRDVEIDTNVIIEGNVSLGNRVKIGTGCVIKNSAIGDD | |
| | CEISPYTVVEDAVLAAACTIGPFARLRPGAVLLEGAHVGNFVEMKKAV | |
| | LGKGSKAGHLTYLGDAAIGDNVNIGAGTITCNYDGANKFTTIIGDDVF | |
| | VGSDTQLVAPVSVGKGATIAAGTTVTRNVGANALAISRVPQTQKEGW | |
| | RRPVKKK | |
| Human Heart | MVDAFLGTWKLVDSKNFDDYMKSLGVGFATRQVASMTKPTTIIEKNG | 13 |
| Fatty Acid | DILTLKTHSTFKNTEISFKLGVEFDETTADDRKVKSIVTLDGGKLVHLQ | |
| Binding | KWDGQETTLVRELIDGKLILTLTHGTAVCTRTYEKEAGSGSGGMHNNR | |
| Protein | LQLSRLERVYQSEQAEKLLLAGVMLRDPARFDLRGSLTHGRDVEIDTN | |
| | VIIEGNVSLGNRVKIGTGCVIKNSAIGDDCEISPYTVVEDAVLAAACTIG | |
| | PFARLRPGAVLLEGAHVGNFVEMKKAVLGKGSKAGHLTYLGDAAIGD | |
| | NVNIGAGTITCNYDGANKFTTIIGDDVFVGSDTQLVAPVSVGKGATIAA | |
| | GTTVTRNVGANALAISRVPQTQKEGWRRPVKKK | |
| CyaY Protein | MNDSEFHRLADQLWLTIEERLDDWDGDSDIDCEINGGVLTITFENGSKI | 14 |
| | IINRQEPLHQVVVLATKQGGYHFDLKGDEWICDRSGETFWDLLEQAAT | |
| | QQAGETVSFRGSGSGSGGMHNNRLQLSRLERVYQSEQAEKLLLAGV | |
| | MLRDPARFDLRGSLTHGRDVEIDTNVIIEGNVSLGNRVKIGTGCVIKNS | |
| | AIGDDCEISPYTVVEDAVLAAACTIGPFARLRPGAVLLEGAHVGNFVE | |
| | MKKAVLGKGSKAGHLTYLGDAAIGDNVNIGAGTITCNYDGANKFTTII | |
| | GDDVFVGSDTQLVAPVSVGKGATIAAGTTVTRNVGANALAISRVPQT | |
| | QKEGWRRPVKKK | |
| DFFA-Like | MGTPRARPCRVSTADRKVRKGIMAHSLEDLLNKVQDILKLKDKPFSL | 15 |
| Effector C | VLEEDGTIVETEEYFQALAKDTMFMVLLAGAKWKPGSGSGGMHNNR | |
| | LQLSRLERVYQSEQAEKLLLAGVMLRDPARFDLRGSLTHGRDVEIDTN | |
| | VIIEGNVSLGNRVKIGTGCVIKNSAIGDDCEISPYTVVEDAVLAAACTIG | |
| | PFARLRPGAVLLEGAHVGNFVEMKKAVLGKGSKAGHLTYLGDAAIGD | |
| | NVNIGAGTITCNYDGANKFTTIIGDDVFVGSDTQLVAPVSVGKGATIAA | |
| | GTTVTRNVGANALAISRVPQTQKEGWRRPVKKK | |
| TDRD2 | MGSRSLQLDKLVNEMTQHYENSVPEDLTVHVGDIVAAPLPTNGSWYR | 16 |
| | ARVLGTLENGNLDLYFVDFGDNGDCPLKDLRALRSDFLSLPFQAIECS | |
| | GSGSGGMHNNRLQLSRLERVYQSEQAEKLLLAGVMLRDPARFDLRGS | |
| | LTHGRDVEIDTNVIIEGNVSLGNRVKIGTGCVIKNSAIGDDCEISPYTVV | |
| | EDAVLAAACTIGPFARLRPGAVLLEGAHVGNFVEMKKAVLGKGSKAG | |
| | HLTYLGDAAIGDNVNIGAGTITCNYDGANKFTTIIGDDVFVGSDTQLV | |
| | APVSVGKGATIAAGTTVTRNVGANALAISRVPQTQKEGWRRPVKKK | |

In some cases, a linker can be used to attach one or more proteins of interest to a self-assembling protein. For example, small linkers can include glycine-serine repeats, glycine-glycine repeats, and a plurality of cysteine residues. A linker can be any appropriate length. In some cases, a linker can include about 1 amino acid to about 300 amino acids (e.g., about 2 amino acids to about 250 amino acids, about 3 amino acids to about 200 amino acids, about 4 amino acids to about 300 amino acids, or about 5 amino acids to about 250 amino acids). For example, a linker can include about 6 to about 8 amino acid residues.

In some cases, a protein of interest can be detectably labeled. Detectable labels include, for example, a histidine tag (e.g., six H residues), fluorescent proteins (e.g., green fluorescent protein (GFP), red fluorescent protein (RFP), yellow fluorescent protein (YFP), fluorescein maleimide (FM), and Alexa Fluor® dyes), and fluorescent quenchers. In cases where a protein of interest includes a binding domain, a detectable label also can be attached to one or more binding targets. In some cases, a protein of interest including a binding domain can have a known binding target, and a detectable label can be attached to the known binding target. For example, a protein of interest can be a Spycatcher protein (SEQ ID NO: 6) which covalently binds a 13-residue Spytag (AHIVMVDAYKPTK; SEQ ID NO: 17). In some cases, the binding target of a protein of interest including a binding domain can be unknown, and one or more detectable labels can be attached to one or more potential binding targets. For example, a different detectable label can be attached to each potential binding target. In some cases, a linker can be used to attach two proteins (e.g., to attach one or more proteins of interest to a self-assembling protein, or to attach a detectable label to a protein of interest).

As will be understood by a skilled person, one or more of the parameters described herein (e.g., self-assembling protein sequence, linker length, linker composition, chosen fusion terminus, expression vector, expression system, and/or expression temperature) can be optimized to achieve the desired 2D protein array (e.g., a 2D protein array presenting a particular protein of interest).

This document also provides nucleic acids encoding self-assembling proteins that can form oligomeric protein unit cells which in turn form 2D protein arrays described herein as well as constructs for expressing nucleic acids encoding self-assembling proteins provided herein. The nucleic acids sequence encoding self-assembling proteins described herein can include RNA, DNA, or any combination thereof. Such nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals.

### Methods of Making a 2D Protein Array

A 2D protein array provided herein can be made by any appropriate method. In some cases, self-assembling proteins can be expressed by a suitable expression system. A suitable expression systems can be a cell-based system (e.g., bacterial systems or eukaryotic systems) or a cell-free system (e.g., in vitro). For example, self-assembling proteins can be expressed by a bacterial (e.g., Escherichia coli) system.

Self-assembling proteins can be expressed at any appropriate temperature. In some cases, self-assembling proteins can be expressed at ambient or room temperature (e.g., about 37° C.). In some cases, self-assembling proteins can be expressed at temperature lower than room temperature (e.g., lower than about 37° C., lower than about 30° C., lower than about 24° C., lower than about 20° C., lower than about 16° C., lower than about 10° C. or lower than about 4° C.). For example, self-assembling proteins can be expressed at about 16° C.

Self-assembling proteins expressed in a cell-based system can be extracted from the cells by any suitable method. In some cases, the cells containing the expressed self-assembling proteins can be disrupted (e.g., by repeated freezing and thawing, sonication, homogenization by high pressure (such as with a french press), homogenization by grinding (such as with a bead mill), and permeabilization by detergents (e.g. Triton X-100) and/or enzymes (e.g. lysozyme)) in order to extract the cellular contents, including the expressed self-assembling proteins. In some cases, proteins, including the expressed self-assembling proteins, can be separated from the cell debris using, for example, centrifugation. For example, proteins (including the expressed self-assembling proteins) and other soluble compounds can remain in the supernatant following centrifugation. In some cases, proteins, including the expressed self-assembling proteins, can be isolated from the cell lysate using, for example, protein precipitation. For example, proteins (including the expressed self-assembling proteins) can be precipitated out of a cell lysate using, for example, precipitation with ammonium sulphate.

Self-assembling proteins can be purified using any suitable technique. Examples of protein purification techniques include pH graded gel, ion exchange column, size exclusion chromatography, sodium dodecyl sulfate-polyacrylamide gel electrophoresis (SDS-PAGE), 2D-PAGE, high performance liquid chromatography, and reversed-phase chromatography. In some cases, a self-assembling protein can include a detectable label (e.g., a His-tag) to facilitate purification. In some cases, a 2D protein array can be made use other appropriate technologies.

Self-assembling proteins will naturally assemble themselves into oligomeric protein unit cells that then naturally assemble themselves into a 2D protein array. Self-assembling proteins can self-interact to form an oligomeric protein unit cell intracellularly (e.g., within a living cell) or extracellularly (e.g., in vitro). Oligomeric protein unit cells also can form a 2D protein array described herein intracellularly or extracellularly. As used herein, intracellular assembly may also be referred to as in vivo assembly.

Without being bound by theory, it is believed that successfully designing a 2D protein array presenting a protein of interest on its surface is a balance of the space afforded by the oligomeric unit cell sizes of the designed arrays (˜5-12 nm) and the size (e.g., molecular weight) of the self-assembling protein.

### Methods of Using a 2D Protein Array

This document also provides methods for using 2D protein arrays provided herein. For example, 2D protein arrays provided herein can be used in biotechnology applications.

In some cases, a 2D protein array provided herein can be used determining a 3D structure of a protein of interest (e.g., a protein having an unknown 3D structure). For example, methods of determining a 3D structure of a protein of interest can include providing a plurality of self-assembling fusion proteins having the protein of interest fused to a self-assembling protein provided herein. Under appropriate conditions, the self-assembling fusion proteins will interact with each other to form a plurality of oligomeric protein unit cells described herein. Such oligomeric protein unit cells then interact with each other to form a 2D protein array presenting the protein of interest on its surface. The 3D structure of a protein of interest being presented on the surface of a 2D protein array can then be determined. In cases where the protein of interest has a binding partner, methods provided herein can also be used to determine the 3D structure of a protein complex (e.g., a protein of interest bound to its binding partner). Suitable techniques for determining the 3D structure of a protein or a protein complex include, for example, X-ray crystallography, NMR spectroscopy, and dual polarization interferometry.

In some cases, 2D protein arrays provided herein can be used to evaluate (e.g., characterize) protein-protein interactions (e.g., stable interactions vs. transient interactions, spatial and/or temporal interactions). For example, a 2D protein array can be used to characterize a binding domain in a protein of interest and/or to identify one or more binding targets of a protein of interest. A binding target can have any function on the protein of interest. For example, a binding target can be an inhibitor, or an agonist. Methods of determining a binding partner of a protein of interest can include providing a plurality of self-assembling fusion proteins having the protein of interest fused to a self-assembling protein provided herein. Under appropriate conditions, the self-assembling fusion proteins will interact with each other to form a plurality of oligomeric protein unit cells described herein. Such oligomeric protein unit cells then interact with each other to form a 2D protein array presenting the protein of interest on its surface. Methods of determining a binding partner of a protein of interest also can include providing a plurality of potential binding targets. Interactions (e.g., binding) between the protein of interest and a potential binding target, as well as certain binding characteristics (e.g., interaction stability, binding affinities, kinetics, spatial proximity, and time course of the interaction), can be determined using any appropriate technique. Suitable techniques include, for example, fluorescence resonance energy transfer (FRET). In cases where FRET is used, a protein of interest can be labeled with a first detectable label, and one or more potential binding targets can be labeled with a second detectable label. In some cases, the first and second detectable labels can be fluorescent proteins having different excitation/emission spectrums. For example, a protein of interest can be labeled with GFP and one or more potential binding targets can be labeled with FM, or a protein of interest can be labeled with, for example, Alexa Fluor® 488 and one or more potential binding targets can be labeled with Alexa Fluor® 647. In some cases, the first detectable label can be a fluorescent protein and the second detectable label can be a fluorescent quencher.

The invention will be further described in the following examples, which do not limit the scope of the invention described in the claims.

### EXAMPLES

### Example 1

### Design of Ordered Two-Dimensional Arrays Mediated by Noncovalent Protein-Protein Interfaces

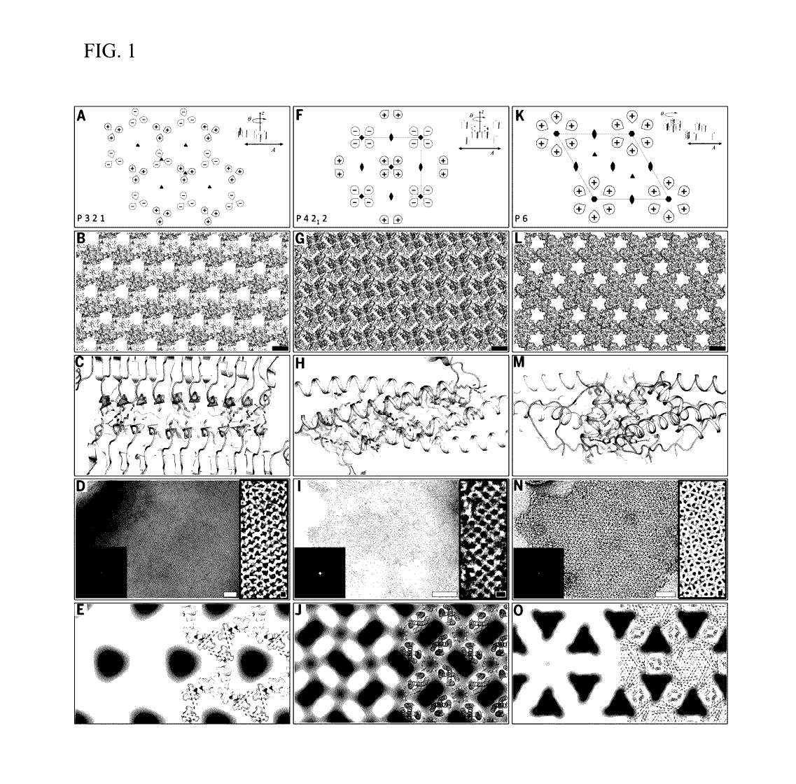

Ordered two-dimensional arrays mediated by designed protein-protein interfaces stabilized by extensive non-covalent interactions were designed. Symmetric arrays were focused on as symmetry reduces the number of distinct protein interfaces required to stabilize the lattice. There are seventeen distinct ways (layer groups) in which three-dimensional objects can come together to form periodic two-dimensional layers (Nannenga et al., “Overview of electron crystallography of membrane proteins: crystallization and screening strategies using negative stain electron microscopy.” Coligan et al. (Eds.) Current Protocols in Protein Science Chapter 17, Unit 17 15 (2013)). In some layer groups there are only two unique interfaces between identical subunits, in others, three or four. Layer groups involving only two unique interfaces, and building blocks with internal point symmetry (which already contain one of the two required interfaces) were focused on leaving only one unique interface to be designed to form the two-dimensional array. Eleven of the seventeen layer groups have two unique interfaces; we focused here on six of these eleven groups involving cyclic rather than dihedral point groups because there are considerably more cyclic oligomers than dihedral oligomers in the Protein Data Bank (PDB) that can serve as building blocks. The six layer groups with two unique interfaces that can be built from cyclic oligomers are P 2 21 21 (from C2 building blocks), P 3 and P 3 2 1 (from C3 building blocks), P 4 and P 4 21 2 (from C4 building blocks), and P 6 (from C6 building blocks). The different groups have different numbers of degrees of freedom describing the placement of an object with cyclic symmetry in the lattice, for example for P 3 2 1 (FIG. 1a) and P 4 21 2 (FIG. 1f), there are three degrees of freedom, whereas for P 6 (FIG. 1k) there are only two.

Symmetric docking in Rosetta was used to search for placements of cyclic oligomers into each of the six layer groups with shape complementary interfaces between different oligomer copies. The docking scoring function consisted of a soft sphere model of steric interactions and a simple measure of the designable interface area: the number of interface Cβs within 7 Å. For each cyclic oligomer in each layer group, ˜20 independent Monte Carlo docking trajectories were carried out starting from placements of 6-9 copies of the oligomer with its symmetry axis aligned with the corresponding symmetry axes of the layer group (for example, trimers were placed on the three-fold symmetry axes indicated by the triangles in FIG. 1A, tetramers on the four-fold symmetry axes indicated by squares in FIG. 1F, and hexamers on the six-fold symmetry axes indicated by hexagons in FIG. 1K). In the Monte Carlo docking simulations, the degrees of freedom sampled were those compatible with the layer group (FIGS. 1A, F, and K right), and hence the layer group symmetry was preserved throughout the calculations.

The most shape complementary (largest number of contacting residues with fewest clashes) solutions from the trajectories were selected and Rosetta sequence design calculations were carried out to generate well packed low energy interfaces between oligomers. Monte Carlo searches were carried out over all amino acid identities and side chain rotamer states for residues near the newly formed interface between oligomers optimizing the Rosetta all atom energy of the entire complex. Following this sequence design step, the energy was further minimized with respect to the side chain torsion angles of residues near the interface and the symmetric degrees of freedom of the layer group. Finally, the resulting lattice models were filtered based on the shape complementarity of the designed interface (>0.5), surface area of the designed interface (>400 Å per monomer), buried unsatisfied hydrogen bonds introduced at the new interface (<4 using a 1.4 Å solvent accessibility probe), and predicted ΔΔG of complex formation (<−10 Rosetta energy units per subunit). The filters were adjusted for each layer group such that approximately 200 designed sequences passed the filters (sample Rosettascripts files accompany the supplementary material). Following further sequence optimization (King et al., Nature 510, 103-108 (2014); Nivon et al., PloS one 8, e59004 (2013)), models passing the filters were manually inspected, and 62 designs were selected for experimental characterization; 16 for P 2 21 21, 2 for P 3, 10 for P 3 2 1, 16 for P 4, 3 for P 4 21 2 and 15 for P 6.

### Materials and Methods

### Computational Design

2D layers were designed that consisted of a native complex with cyclic symmetry, such that one designed interface would lead to self-assembling two-dimensional lattices. This leads to 7 possible layer groups: C 2 1 1 and P 2 21 21 (from C2 building blocks), P 3 and P 3 2 1 (from C3 building blocks), P 4 and P 4 21 2 (from C4 building blocks), and P 6 (from C6 building blocks). Additional layer groups (C 2 2 2, P 3 1 2, P 4 2 2, and P 6 2 2) are possible starting from native complexes with dihedral symmetry, but the relatively low availability of crystal structures of such complexes led us to focus on only starting structures with cyclic symmetry. The remaining six layer groups require the design of more than one interface starting from a point-symmetric building block.

The Protein Data Bank (PDB) was searched for native complexes with the appropriate symmetry. Structures with a biological unit containing 2, 3, 4, or 6 chains with identical (or nearly identical) sequences that deviated from perfectly symmetric by less than 2 Å RMSD were identified. The data was further limited to complexes with an asymmetric unit between 100 and 400 residues, and was trimmed to reduce redundancy by throwing out structures with >90% sequence identity; due to the large number of native C2 complexes, this was reduced to 30% for C2-symmetric building blocks. This resulted in 2929 native C2 complexes, 290 native C3 complexes, 74 native C4 complexes, and 26 native C6 complexes.

Symmetric docking in Rosetta was used in order to find designable configurations of each of the point-symmetric complexes into 2D layers. A symmetry definition file was generated that modeled the inner point symmetric complex as well as the 6 or 8 complexes immediately surrounding it. During docking, the rigid-body perturbations were limited to those that maintained the configuration of the native point symmetric complexes. This led to only 2 (P 3, P 4 and P 6), 3 (P 3 2 1 and P 4 21 2), or 4 (P 2 21 21) rigid-body degrees of freedom that are allowed to optimize during each docking trajectory. During docking, a scoring function with only two terms was used: the first modeled sterics using a soft sphere model; the second provides a rough estimate of designable interface area by counting the number of interface Cβs within 7 Å distance. For each starting model, ˜20 independent Monte Carlo docking trajectories were carried out from each starting point (with more for C6 building blocks and fewer for C2 building blocks). Each resulting model was then designed.

The design methodology employed was similar to that used for the design of closed symmetric complexes in Rosetta (King et al., Science 336, 1171-1174 (2012); King et al., Nature 510, 103-108 (2014)). All residues near to the interface and not part of the native interface had their residue identity and rotameric state changed in a Monte Carlo search optimizing the Rosetta energy of the entire complex. Each model then had side chain torsions as well as the symmetric degrees of freedom simultaneously minimized with respect to the energy function. Finally, these models were filtered using several different criteria: shape complementarity of the designed interface (>0.5), surface area of the designed interface (>400 Å per monomer), buried unsatisfied hydrogen bonds (Hendsch et al., Biochemistry 35, 7621-7625 (1996)) introduced at the new interface (<4 using a 1.4 Å solvent accessibility probe size), and predicted ΔΔG (Kellogg et al., The journal of physical chemistry. B 116, 11405-11413 (2012)) of complex formation (<−10 energy units per subunit). The filters were adjusted for each layer group such that approximately 200 designed sequences passed the filters. Structures passing the filters were manually inspected, and then subject to additional automatic (Nivon et al., PloS one 8, e59004 (2013)) and manual optimization. All designs were visualized in PyMOL (The PyMOL Molecular Graphics System, Version 1.7.2, Schrödinger, LLC (pymol.org)). The filter scores for the four designs that yielded crystals are presented in Table 4.

TABLE 4
Final Rosettascripts filter scores for p3Z_11, p3Z_42,
p4Z_9 and p6_9H.

| | | | | Unsatisfied |
|---|---|---|---|---|
| Design | ΔΔG | Mutations | Shape Complementarity | Polar Residues |
| p3Z_11 | −13.34 | 9 | 0.682 | 1 |
| p3Z_42 | −20.8 | 11 | 0.634 | 2 |
| p4Z_9 | −16.12 | 10 | 0.648 | 2 |
| p6_9H | −15.83 | 12* | 0.73 | 0 |

*An additional mutation (A29D) was introduced during gene synthsis

All scripts and source code used in computational layer design has been included in Rosetta3 including source code, available at rosettacommons.org. Any weekly release of Rosetta after May 1, 2015 can be used for the material in this study.

All the necessary inputs for replicating the calculations performed in this manuscript—including native PDB files, symmetry definition files, RosettaScripts inputs, and PDB files of the final designs of four crystals highlighted in this paper accompany the online version of this manuscript. Sequence design also made use of previously published optimization scripts. *note* Scripts contain a %% nbblock %% flag—this is equivalent to the cyclic symmetry of the associated scaffold (e.g. 2 for C2, 3 for C3, 4 for C4 and 6 for C6) *note*

Finally, a perl script is available that allows the creation of symmetry definition files for any of the seven C-symmetry compatible layer groups described in the manuscript. The script handles symmetrization of nearly-symmetric inputs as well as generation of the inputs needed for Rosetta to construct the lattice. It can be found in the Rosetta directory path ‘apps/public/symmetry/make_Pn_tiling.pl’.

### Design Sequences

Genes were purchased from either Gen9 (http://www.gen9bio.com/) (including p6_9H) or Genescript (http://www.genscript.com/) (including p3Z_11, p3Z_42 and p4Z_9). Genes purchased from Gen9 were cloned into pet15 (Ampicillin/Carbenicillin resistant) expression vector. Genescript genes were purchased pre-inserted into pet29b (Kanamycin resistant) expression vector. A mutation (A29D) was introduced during gene synthesis to p6_9 and was retained in this study. Wildtype sequences are shown in Table 5 below.

TABLE 5
Wildtype self-assembling protein sequences.

| | amino acid sequence | SEQ ID NO: |
|---|---|---|
| p3Z_11 | MEEVVLITVPSEEVARTIAKALVEERLAACVNIVPGLTSIYRWQGEV | 18 |
| | VEDQELLLLVKTTTHAFPKLKERVKALHPYTVPEIVALPIAEGNREY | |
| | LDWLRENTG | |
| p3Z_42 | MHNNRLQLSRLERVYQSEQAEKLLLAGVMLRDPARFDLRGTLTHG | 19 |
| | RDVEIDTNVIIEGNVTLGHRVKIGTGCVIKNSVIGDDCEISPYTVVED | |
| | ANLAAACTIGPFARLRPGAELLEGAHVGNFVEMKKARLGKGSKAG | |
| | HLTYLGDAEIGDNVNIGAGTITCNYDGANKFKTIIGDDVFVGSDTQ | |
| | LVAPVTVGKGATIAAGTTVTRNVGENALAISRVPQTQKEGWRRPV | |
| | KKK | |
| p4Z_9 | MEAVRAYELQLELQQIRTLRQSLELKMKELEYAEGIITSLKSERRIY | 20 |
| | RAFSDLLVEITKDEMEHIERSRLVYKREIEKLKKREKEIMEELSKLR | |
| | APLS | |
| p6_9H | FQGPLGSHMTISPKEKEKIAIHEAGHALMGLVSDDDDKVHKISIIPR | 21 |
| | GMALGVTQQLPIEDKHIYDKKDLYNKILVLLGGRAAEEVFFGKDGI | |
| | TTGAENDLQRATDLAYRMVSMWGMSDKVGPIAIRRVANPFLGGM | |
| | TTAVDTSPDLLREIDEEVKRIITEQYEKAKAIVEEYKEPLKAVVKKL | |
| | LEKETITCEEFVEVFKLYGIELKDKCKKEELFDKDRKSEENKELKSE | |
| | EVKEEVV | |

Mutagenesis (p6_9 and p6_9H)

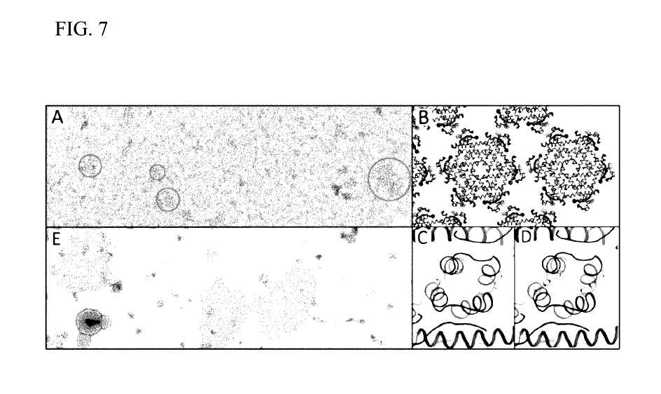

Oligonucleotides containing the mutations required were ordered from IDT (idtdna.com/). Mutations were made by either the single stranded DNA “Kunkel Mutagenesis” method or by quickchange mutagenesis using pFU Ultra II DNA polymerase (Agilent) and dNTP's (Thermo Scientific). FIG. 7 and Table 6 highlight the mutants made on design p6_9 (precursor to p6_9H). All mutated sequences were verified by either Genewiz (genewiz.com/) or internally at Janelia Research Center's molecular biology core.

TABLE 6
Mutagenesis of p6_9 design (pre-cursor of p6_9H)

| | Sizes of crystals |
|---|---|
| Mutation/s | observed in the pellet |
| Original Design p6_9 (Control) | + |
| A184S | + |
| T203V | + |
| E188R | + |
| E199L | + |
| E188H (p6_9H) | +++ |
| F181R | None observed |
| L193T | + |
| L193T, A198V | + |
| L193T, S189K | + |
| L193T, A198V, S189K | ++ |
| L193T, A198V, S189K, L177E | None observed |
| L193T, A198V, S189K, cut 6xHis | ++ |
| E188H, V200M (p6_9HM) | +++ |
| E188H, F218Y | +++ |
| E188H, D29A | +++ |
| E188H, L193T, A198V | +++ |
| E188H, cut 6xHis | +++ |
| E188H, short construct (p6_9H_KDKCKXX) | ++ |

p6_9H_KDKCKXX Construct

A new construct was made from p6_9H, where 33 C-terminal amino acid residues (including 6×HIS) not used at the protein-protein interface and not having structural information in the original WT crystal structure were removed in order to check protein stability, called p6_9H_KDKCKXX. This significant (˜15% including 6×His) removal of residues from the protein did not result in breaking the arrays. Protein stability was reduced however with stacked 2D crystals viewed in a similar ratio as single layered sheets suggesting these residues are required for the original C6 scaffold stability.

### Protein Expression

All proteins were expressed by first transforming all purified plasmid DNA into BL21 (DE3) E. coli cells. Culture was grown in LB medium with the addition of either 50 mg L⁻¹ Kanamycin (Sigma) (p3Z_11, p3Z_42 and p4Z_9) or 100 mg L-1 Ampicillin (Fisher Scientific) (p6_9H) until OD600 ˜0.4 was reached at 37° Celsius. Expression was induced by the addition of 1 mM IPTG (Sigma) and allowed to continue for 4 hours at 37° Celsius. For p3Z_42 cryo-EM sample, expression was induced with 0.1 mM IPTG for ˜19 hours at 16° Celsius after reaching OD600 ˜0.2-0.4 at 37° Celsius. All culture was centrifuged to separate and remove the media from the cells and the cells frozen at −20° Celsius. Cells were re-suspended in Lysis buffer (25 mM Tris pH 8.0, 150 mM NaCl) with 1 mM DTT (Acros) (p3Z_11, p3Z_42 and p6_9H) or without DTT (p4Z_9). Protein was recovered by the use of either a Sonicator (Fisher Scientific) or a Microfluidizer (microfluidics) after the addition of either 1 mM PMSF (Fisher Scientific) or recommended amount of dissolved EDTA-free protease inhibitor tablet/s (Thermo Scientific). Soluble supernatant was separated from insoluble pellet material by ultracentrifugation at 12,000×G using a Ti50.2 or Ti70 rotor (Beckman Coulter) at 4° Celsius for 30 minutes. Pellet material was re-suspended in lysis buffer and kept at 4° Celsius. All expressions were verified by SDS-PAGE (BioRad).

In Vitro Expression (p3Z_42)

An Expressway (Invitrogen) cell-free protein expression kit was used as recommended with purified p3Z_42 plasmid DNA and left for the maximum time recommended for expression (4 hours) at 37° Celsius. Negative-stain sample grids were made using the expression solution directly without purification or separation of material and visualized for crystal growth. Expression was also verified by SDS-PAGE as above.

Protein Denaturing and Refolding (p4Z_9)

Frozen cell pellets made from expressed p4Z_9 cells grown at 37° Celsius were resuspended in lysis buffer (25 mM Tris pH 8.0, 150 mM NaCl) supplemented with EDTA-free protease inhibitor tablets (Thermo Scientific) and lysed by use of a Microfluidizer (Microfluidics). The resulting solution was spun in a Ti50.2 or Ti70 ultracentrifuge rotor (Beckman Coulter) for 30 minutes at 12,000×g at 4° Celsius. Supernatant was discarded and pellet material was re-suspended in denaturing buffer (6M Guanidine HCL, 25 mM Tris pH 8.0, 150 mM NaCl) and the solution left in a 37° Celsius incubator for 1 hour. The solution was then filtered with 0.22 μm filters (Millipore). Ni-NTA agarose (Qiagen) in denaturing buffer with 20 mM Imidazole were added and the solution allowed to rotate slowly at 4° Celsius for two or more hours or overnight. The solution was then run on a gravity column and the beads washed twice with the same denaturing solution with 20 mM Imidazole. p4Z_9 proteins were then eluted with denaturing buffer with 500 mM Imidazole and concentrated using a 5K MWCO Vivaspin (Sartorius Stedim) column. The solution was then run through a Superdex 200 (10/300) column (GE Healthcare) on a (Biorad) FPLC, pre-equilibrated with denaturing buffer. Pure p4Z_9 was collected by fractionation. Fractions containing protein were pooled and concentrated again as above. Concentrations were verified by Nanodrop (Thermo Scientific) or BCA assay (Thermo Scientific). Purity was verified by SDS-PAGE (Biorad).

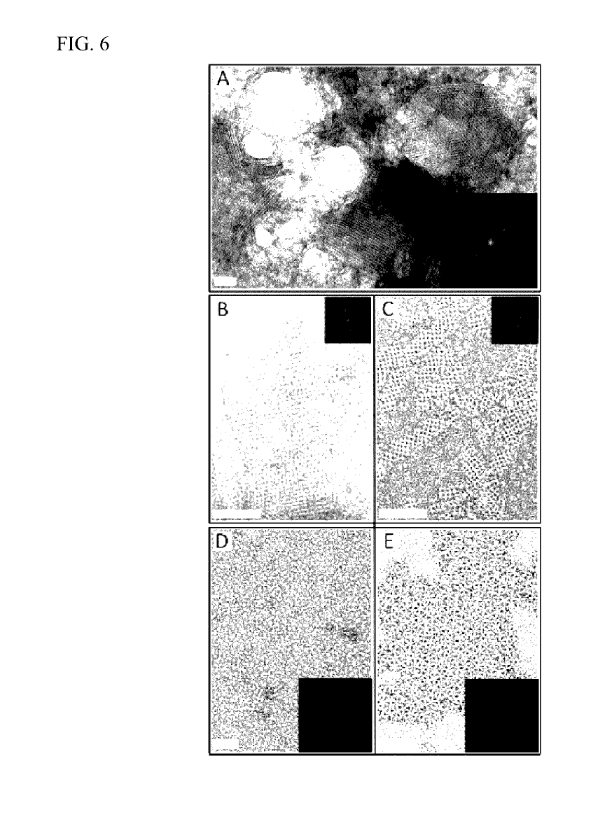

Refolding of p4Z_9 was done using either fast dilution or dialysis. For dilution, the concentrated solution was added to varying amounts of lysis buffer (25 mM Tris pH 8.0, 150 mM NaCl) at 4° Celsius. The solution was then concentrated as above and analyzed by negative-stain EM (Fig. S4b). For dialysis, the denatured solution was injected into a wet dialysis cassette (Thermo Scientific) revolving in a bath of lysis buffer at room temperature and allowed to refold for 1 hour or overnight at 4° Celsius. Re-folded protein was extracted from the dialysis cassette and viewed by negative-stain EM (FIG. 6C).

Protein Purification and In Vitro Assembly (p6_9H)

Supernatant p6_9H was separated from the pellet material and filtered with 0.22 μm filters (Millipore). Ni-NTA agarose (Qiagen) in lysis buffer with 1 mM DTT and 20 mM Imidazole was added to the solution allowed to rotate slowly at 4° Celsius for 2 Hours or more. The solution was then run on a gravity column and beads washed twice with lysis buffer and 1 mM DTT and 20 mM Imidazole for the first wash and 1 mM DTT and 40 mM imidazole for the second. The protein was then eluted with lysis buffer with 1 mM DTT and 500 mM Imidazole. The solution was run on a pre-equilibrated Sephacryl S-300 (26/60) (GE Healthcare) column in a (biorad) FPLC and fractions collected. Fractions were then pooled and concentrated in a 10K MWCO Vivaspin (Sartorius Stedim) column. The protein concentration was determined using a BCA assay (Thermo Scientific) and purity was verified by SDS-PAGE (Biorad) and flash frozen using liquid nitrogen and stored at −80° Celsius. Arrays were not seen at this point and the sample appeared as homogeneous single particles (FIG. 6D). The protein was concentrated to ˜30 mg/mL and extensive arrays were observed after 1 hour incubation at 37° Celsius (FIG. 6E).

### Negative-Stain Electron Microscopy

A drop of 2-3 μL sample was applied on negatively glow discharged, carbon-coated 200-mesh copper grids (Ted Pella, Inc.), washed with Milli-Q Water and stained using 0.75% uranyl formate. Screening was performed on either a 120 kV Tecnai Spirit T12 transmission electron microscope (FEI, Hillsboro, Oreg.) or a 100 kV Morgagni M268 transmission electron microscope (FEI, Hillsboro, Oreg.). Images were recorded on a bottom mount Teitz CMOS 4 k camera system. The contrast of the images was enhanced in Fiji (Schindelin et al., Nature methods 9, 676-682 (2012)) for clarity.

### Projection Maps

Micrographs of negatively stained preparations or of cryo preparations were processed in the MRC suite of programs through the 2dx interface.

### Cryo Electron Microscopy and Motion Corrected Movies

An aliquot of 2 μL of p3Z_42 sample was placed onto a holey carbon grid and plunged into liquid ethane using a FEI vitrobot and cryo transferred onto a cryo microscope under liquid nitrogen temperatures. Samples were viewed on either an FEI Technai F20 using a Teitz 4×4 k camera or an FEI Titan Krios using a K2 camera to record super-resolution movies. All movies were motion corrected using software with a bin of 1. Diffraction data were collected on the FEI Technai F20 operating in diffraction mode and recorded on a Teitz 2×2 k camera and processed in XDP. The contrast of the images was enhanced in Fiji for clarity.

All panels were made using PyMOL, Fiji, and assembled in Adobe Photoshop CS5 (adobe.com).

### Results

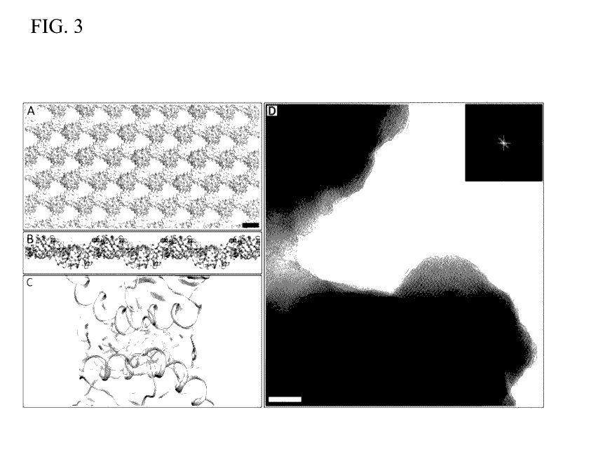

Synthetic genes were obtained for the 62 designs, and the proteins were expressed in the Escherichia coli cytoplasm by using a standard T7-based expression vector. Of the 62 designs, 43 expressed; of these, 18 had protein in the supernatant after clearing the lysate at 12,000×g for 30 minutes, whereas all 43 had protein in the pellet. To investigate the degree of order in the pelleted material, negatively stained samples were examined by electron microscopy (EM). Regular lattices were observed for four of the designs: one formed only stacked 2D layers (FIG. 3), whereas three formed planar arrays. The latter are described in the following sections.

p3Z_11

Design p3Z_11 (P 3 2 1 symmetry) (FIG. 3) was found to make stacked 2D or 3D crystals in vivo. The interface is made up of six interlocking Isoleucine residues flanked by serine-histidine hydrogen bonds on two sides of the anti-parallel interface resulting from the flipped orientation of the trimeric building blocks. The z height between subunits differs from the plane of the crystal by a substantial amount causing the entire 2D assembly to be in a zipper-like motif that is perhaps conducive to the formation of 3D crystals in the small, highly concentrated environment found in vivo.

p3Z_42

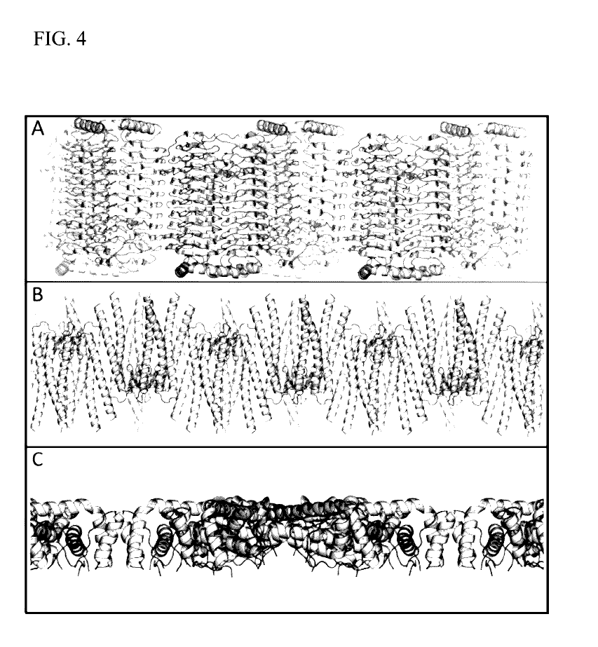

Design p3Z_42 is in layer group P 3 2 1. The rigid body arrangement of the constituent beta-helix trimers in the lattice was identified by Monte Carlo search over the three degrees of freedom of the lattice: the rotation of the trimer around its axis, the lattice spacing, and the z offset of the trimer from the lattice plane (FIG. 1A). In the lattice identified in the Monte Carlo docking calculations, the oligomeric building blocks pack into a dense array (FIG. 1B; the yellow and purple copies are inverted with respect to each other, side view FIG. 4A) stabilized by a large contact surface between adjacent copies with close complementary side chain packing (FIG. 1C) generated in the sequence design calculations.

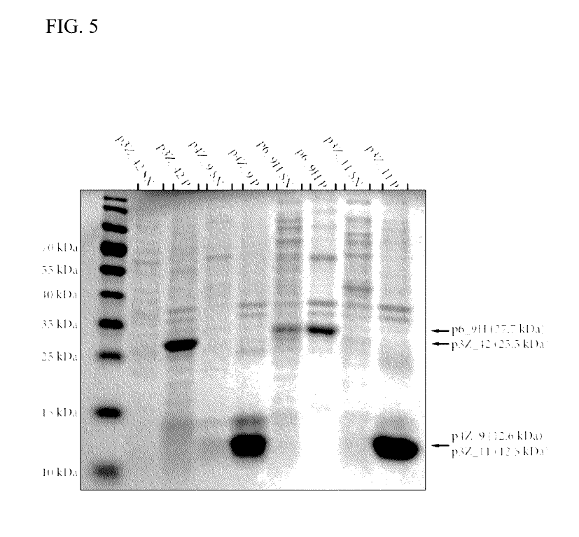

p3Z_42 formed large and very well ordered 2D crystals (FIG. 1D). Very little of the protein produced in E. coli was found in the soluble fraction (FIG. 5), suggesting the vast majority of the expressed protein assembled into the crystalline arrays found in the pellet fraction. At low (16° Celsius) expression temperatures, 2D sheets were obtained (FIG. 1D), while at 37° Celsius, where larger amounts of proteins are produced, large 2D sheets mainly stacked into thick 3D crystals. Higher magnification (FIG. 1D, inset) showed a trigonal lattice similar to that of the design model (compare FIG. 1D (right) with 1B). Fourier transformation of the lattice (FIG. 1D (left)) yielded peaks out to 15 Å resolution; the order in the unstained lattice is probably significantly higher as the negative stain likely limits the observed resolution. A 15 Å projection map (FIG. 1E) back-computed from the Fourier components followed the contour of the designed lattice (FIG. 1E (right)) (unit cell dimensions a=b=85 Å, γ=120°). It is notable that planar crystals of such large size can grow without support within the confines (and with the many cellular obstacles) of an E. coli cell. Cell free expression of this design yielded large ordered 2D crystals similar to those formed in E. coli (FIG. 6A).

p4Z_9

Design p4Z_9 is in layer group P 4 21 2. Search over the three degrees of freedom of the layer group (the rotation around the internal C4 axis, the lattice spacing, and the z offset between adjacent inverted tetramers (FIG. 1F)) yielded the close packed arrangement shown in FIG. 1G (side view FIG. 4B). The designed interface is composed of hydrophobic residues nestled between two alpha helices surrounded by polar residues (FIG. 1H).

p4Z_9 formed crystals up to a micron in width (FIG. 1I) with little of the protein present in the soluble fraction (FIG. 5). Incubation of the pellet material with 6M guanidine and subsequent purification and refolding (by dialysis or fast dilution) yielded crystalline 2D arrays and fibers with the same square packing (FIG. 6B, 6C). Fourier transformation of the negatively stained large in vivo generated 2D lattices yielded peaks out to 14 Å resolution (FIG. 1I (left)). The 14 Å projection map produced by back transformation had distinctive rectangular voids in alternating directions closely matching the design model (FIG. 1J) (unit cell dimensions a=b=56 Å, γ=90°).

p6_9

Design p6_9 is built from alpha helical hexamers in layer group P 6. In this case all oligomers are in the same orientation along the z-axis (perpendicular to the plane in FIG. 1K) and hence there are only two degrees of freedom—the rotation around the six-fold axis and the lattice spacing (FIG. 1K (right)). The shape complementary docking solution (FIG. 1L, side view FIG. 4C)) is composed of four closely associating alpha helices along the two-fold axis of the lattice (FIG. 1M) with two interacting phenylalanines. We also tested a variant, p6_9H, which introduces a hydrogen bond network across the interface (FIG. 1M).

Design p6_9 expressed in E. coli was found in both the supernatant and pellet (FIG. 5). EM investigation revealed that the pellet contained highly ordered single layer 2D hexagonal arrays while the supernatant did not. p6_9H formed even larger arrays (FIG. 1N, FIG. 7, and Table 6). The 2D layers in the pellet were highly ordered with clearly evident hexagonal packing (FIG. 1N). Fourier transformation of the negatively stained arrays (FIG. 1N (left)) yielded peaks out to 14 Å resolution; and the back-computed 14 Å map was again closely consistent with the design model of the array (FIG. 1O; unit cell dimensions: a=b=120 Å, γ=120°). Large arrays were also formed in vitro following concentration of soluble p6_9H purified from the supernatant after lysis of E. coli (FIG. 6D, 6E).

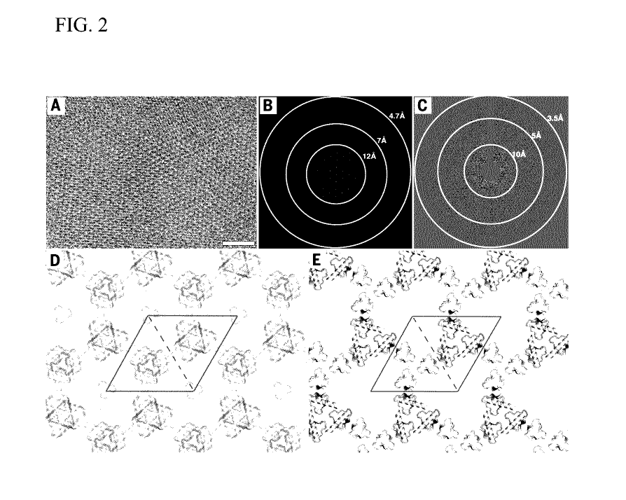

To achieve higher resolution than possible with negatively stained samples, we analyzed designs without stain by electron cryomicroscopy (cryo EM). Analysis of p3Z_42 crystals by cryo EM (FIG. 2A, 2B) and electron diffraction yielded data visible to 3.5 Å resolution (FIG. 2C). The vast majority of crystals diffracted to this resolution in the cryo preparations indicating high long-range order. Movie micrographs of the resulting crystals were also collected, motion corrected and processed in 2dx (25) to yield a projection map at 4 Å resolution in agreement with the design model (FIG. 2, compare panels D and E). To our knowledge, this is highest order observed for a designed macromolecular 2D lattice to date.

### 2D Protein Arrays

Designed planar protein arrays form large planar 2D crystals both in vivo and in vitro that are closely consistent with the design models. Two of the three successes were with layer groups with adjacent building locks in opposite orientations along the z axis; these have the advantage that 1) there is an additional degree of freedom (the z offset) providing more possible packing arrangements for a given oligomeric building block, 2) the interfaces are antiparallel rather than parallel so that in the design calculations opposing residues can have different identities, and 3) inaccuracies in the design calculations that result in deviation from planarity effectively cancel out. On the other hand, designed “polar” arrays with all subunits orientated in the same direction; such as p6_9—have advantages for functionalization as the two sides are distinct and can be addressed separately.

It is notable that, for all three designs, extensive crystalline arrays form unsupported in E. coli and from purified protein in vitro. The coherent arrays can extend up to 1 μm in length but are only 3 to 8 nM thick by design (FIG. 4).

These results show that self-assembling proteins (e.g., p3Z_42, p4Z_9, and p6_9H) can self-assemble into 2D protein arrays, and that the self-assembling proteins can be specifically designed to assemble 2D protein arrays at the near atomic level.

### Example 2

### Atomic Patterning of Proteins and Fluorescent Dyes Using Designed Two-Dimensional Protein Arrays

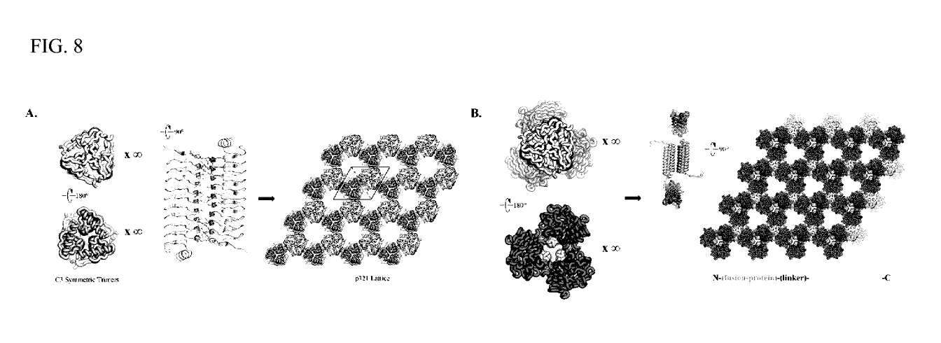

Proteins of interest were genetically fused to the N- or C-terminus of each of the array monomers using small linkers made of Glycine-Serine and Glycine-Glycine repeats (6-8 amino acid residues total), whereby the designed residues will drive self-assembly of both proteins (FIG. 8B). Based on the results obtained in the original study, design p4Z-9 had the smallest unit cell size (˜5 nm repeats) and was made up of very small proteins (˜12 kDa) and design p6-9H was shown to be both slow to form an array in vivo and highly soluble in vitro unless concentrated to a very high concentration. p3Z-42 is made up of large building blocks (˜25 kDa) and was shown to assemble into arrays at a very fast rate, both by in vivo and in vitro expression and would be well suited for fusion arrays.

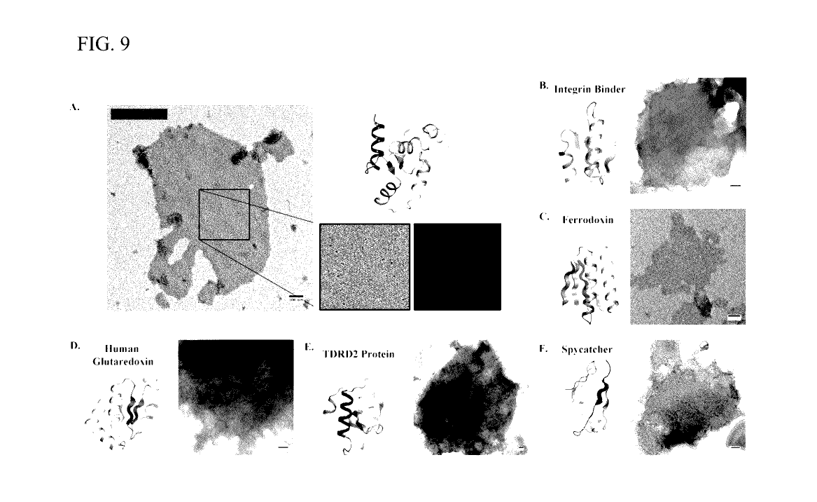

Synthetic genes of each fusion were obtained and protein was expressed in Escherichia coli cells using a standard T7 based expression vector (Table 2). The protein expression was verified by sodium dodecyl sulfate polyacrylamide gel electrophoresis (SDS-PAGE) after separation of the soluble and insoluble cell portions. Samples of observed protein in the cellular pellets were analyzed for array formation by negative-stain Transmission Electron Microscopy (TEM). Fused proteins of Spycatcher, Ferrodoxin and an Integrin binder called av6-3 were shown to make large and well-ordered 2D crystals (FIG. 9A). It is worth noting that 5/7 of the remaining fusions either shared a similar sequence and unknown but potentially similar structures (2 unpublished binding proteins) or were either the same molecular weight or much larger than the p3Z-42 monomer protein (3 proteins between ˜25-35 kDa).

On the basis of these initial hits, the general properties of the proteins that crystallized, specifically molecular weight, were evaluated. 16 further fusions were identified based either on smaller molecular weight sizes (13 proteins between 9 and 13 kDa) or other important targets close to this molecular weight range (3 proteins between 14 and 17 kDa). These second screen fusions were genetically fused and checked for array formation as before. 9/16 of the proteins were found to form 2D arrays of varying sizes, some larger than the original design alone, straight out of the Escherichia coli insoluble pellet material (FIG. 9B). In total, 12 brand new 2D crystals were successfully created, including: the human variant of the fatty-acid binding protein, Calmodulin (p3Z-42-Calmodulin), Human Glutaredoxin and Human Acylphosphatase from the second set of hits (FIG. 9B).

In order to further characterize the fusion proteins, p3Z-42-Calmodulin was analyzed using Cryo-EM. p3Z-42-Calmodulin was chosen as the average 2D crystals observed by negative-stain EM had hundreds or thousands of unit cells. Some p3Z-42-Calmodulin crystals also reached >1 μm in size (FIG. 9A) and were highly ordered, with many spots observed by Fourier Transformation (FIG. 9A). Calmodulin is an important secondary messenger in the cell. Re-suspended pellet material was used and frozen using liquid ethane to form grids with a thin layer of vitrified ice. High-resolution movies were collected and motion corrected micrographs were observed to contain highly ordered 2D crystals. When Fourier transforms were calculated, sharp spots were observed (FIGS. 9A-9F). Using these micrographs, we were able to calculate high-resolution, projection maps to compare to the previously reported projection map for p3Z_42. This result highlights not only that the Calmodulin fusion forms 2D crystals different from the original p3Z-42 array, but also that they are highly ordered just by having a small fusion linker without additional anchors or modifications.

The Spycatcher protein has a unique and highly customizable property, whereby a 13-residue peptide, called Spytag, is able to covalently and irreversibly bind to Spycatcher in vitro. This new p3Z-42-Spycatcher array (p3Z-42-SC) is therefore an array capable of binding other proteins or peptides expressing the Spytag peptide in vitro with strong covalent interactions.

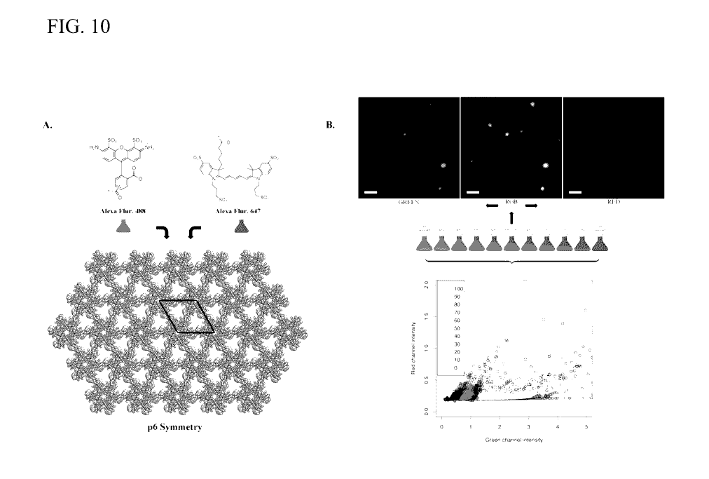

Pure Spytagged-fused superfolder variant of Green Fluorescent Protein (SFGFP) was added straight to the pellet material of p3Z-42-SC and covalent binding to the array could be observed with a band shift by SDS-PAGE. A 19-residue version of Spytag that contained a short Glycine and Serine motif linker with a single cystine at the C-terminus was attached to a fluorescent dye, fluorecine maleimide (FM) by the reaction of the maleimide to the sulfhydryl group of the cystine and this new Spytag-FM was added as with Spytag-SFGFP (FIG. 10A) and was observed to bind by SDS-PAGE. When p3Z-42-SC-Spytag-FM was excited at ˜488 nm, a signal could be observed much stronger than that of labeled single proteins.

Spytag-FM and Spytag-SFGFP were added to a 2D p3Z-42-SC array in varying rations (FIG. 10B, middle panel). Spycatcher-Spytag binding to both Spytag labeled with Alexa Fluor® 488 and/or Alexa Fluor® 647 was detected using FRET (FIG. 10B, top panel). The emission intensity for each label (FIG. 10B, bottom panel) illustrated proportional increases, showing consistent transfer of energy in the labeled protein array.

This study reports 12 completely new and different 2D protein arrays. To our knowledge, this is the first known case of 2D arrays of biological material forming in vivo purely by genetic fusion to self-assembling protein arrays mediated by noncovalent interfaces. The ability to potentially form 2D crystals from most small monomeric proteins and patterning fluorescent dyes should enable new approaches in nanotechnology, bioengineering, structural biology and fluorescent microscopy.

These results show that 2D protein arrays presenting a protein of interest can be formed by intracellularly by genetically fusing the protein of interest to a self-assembling protein. These results also show that a designed 2D protein array presenting a protein of interest can be used to detect binding of a ligand to the protein of interest.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A two-dimensional (2D) protein array comprising:
a plurality of oligomeric protein unit cells, wherein each oligomeric protein unit cell comprises at least one axis of rotational symmetry, and wherein each oligomeric protein unit cell comprises a plurality of self-assembling proteins;
wherein said plurality of oligomeric protein unit cells interact with one another at one or more symmetrically repeated protein-protein interfaces.

2. The 2D protein array of claim 1, wherein said axis of rotational symmetry is cyclic or dihedral.

3. The 2D protein array of claim 1, wherein said one or more symmetrically repeated protein-protein interfaces comprises two, three, or four symmetrically repeated protein-protein interfaces.

4. The 2D protein array of claim 1, wherein said oligomeric protein unit cell is selected from the group consisting of a dimeric protein unit cell, a trimeric protein unit cell, a tetrameric protein unit cell, a pentameric protein unit cell, or a hexameric protein unit cell.

5. The 2D protein array of claim 1, wherein said at least one axis of rotational symmetry comprises the z axis.

6. The 2D protein array of claim 1, wherein said oligomeric protein unit cell comprises a surface area of greater than 400 Å2.

7. The 2D protein array of claim 1, wherein said oligomeric protein unit cell comprises a shape complementarity of about 0.1 Sc to about 10 Sc.

8. The 2D protein array of claim 7, wherein said oligomeric protein unit cell comprises a shape complementarity of about 0.5 Sc to about 1.8 Sc.

9. The 2D protein array of claim 1, wherein said plurality of self-assembling proteins comprises a self-assembling protein selected from the group consisting of: p3Z_11 (SEQ ID NO: 1); p3Z_42 (SEQ ID NO: 2); p4Z_9 (SEQ ID NO: 3); p6_9H (SEQ ID NO: 4); or p6_9H_KDKCKXX (SEQ ID NO: 5).

10. The 2D protein array of claim 1, wherein said plurality of self-assembling proteins comprises a self-assembling protein about 25 to about 500 amino acids in length.

11. The 2D protein array of claim 10, wherein said self-assembling protein is about 200 to about 250 amino acids in length.

12. The 2D protein array of claim 1, wherein at least one of said plurality of self-assembling proteins is a self-assembling fusion protein.

13. The 2D protein array of claim 12, wherein said self-assembling fusion protein comprises a self-assembling protein fused to a protein of interest.

14. The 2D protein array of claim 13, wherein said self-assembling fusion protein further comprises a linker between said self-assembling protein and said protein of interest.

15. The 2D protein array of claim 14, wherein said linker comprises a glycine-glycine or a glycine-serine.

16. The 2D protein array of claim 13, wherein said protein of interest is a protein with an unknown three dimensional (3D) structure.

17. The 2D protein array of claim 13, wherein said protein of interest is a protein with an unknown binding partner.

18. The 2D protein array of claim 1, wherein said interaction between said oligomeric protein unit cells is a non-covalent interaction.

19. The 2D protein array of claim 1, wherein said 2D protein array has a thickness of about 0.1 nM to about 100 nM.

20. The 2D protein array of claim 19, wherein said 2D protein array has a thickness of about 3 nM to about 8 nM.

21. The 2D protein array of claim 1, wherein said 2D protein array has a length of about 0.05 μm to about 5 μm.

22. The 2D protein array of claim 21, wherein said 2D protein array has a length of about 1 μm.

23. A method of assembling a two-dimensional (2D) protein array comprising:
providing a plurality of self-assembling proteins under conditions that allow said self-assembling proteins to interact with one another to form a plurality of oligomeric protein unit cells, wherein each oligomeric protein unit cell comprises at least one axis of rotational symmetry;
wherein said plurality of oligomeric protein unit cells interact with each other at one or more symmetrically repeated protein-protein interfaces to form said 2D protein array.

24. The method of claim 23, wherein said providing comprises expressing said plurality of self-assembling proteins from a cell-based expression system.

25. The method of claim 24, wherein said cell-based expression system is a bacterial expression system.

26. The method of claim 25, wherein said bacterial expression system is an Escherichia coli expression system.

27. The method of claim 20, wherein said 2D protein array is formed intracellularly.

28. A method for determining a three dimensional (3D) structure of a protein of interest, said method comprising:
providing a plurality of self-assembling fusion proteins under conditions that allow said self-assembling fusion proteins to interact with one another to form a plurality of oligomeric protein unit cells, wherein at least one of said self-assembling fusion proteins comprises a self-assembling protein fused to the protein of interest, wherein each of said plurality of oligomeric protein unit cells comprises at least one axis of rotational symmetry;
wherein said plurality of oligomeric protein unit cells interact with each other at one or more symmetrically repeated protein-protein interfaces to form a 2D protein array, wherein said 2D protein array presents the protein of interest on its surface; and
determining the 3D structure of the protein of interest present on the surface of the 2D protein array.

29. The method of claim 28, wherein said determining comprises X-ray crystallography, NMR spectroscopy, or dual polarisation interferometry.

30. A method for determining a binding partner of a protein of interest, said method comprising:
providing a plurality of self-assembling fusion proteins, wherein each of said self-assembling fusion proteins comprises a self-assembling protein fused to the protein of interest, under conditions that allow said self-assembling fusion proteins to interact with each other to form a plurality of oligomeric protein unit cells, wherein each oligomeric protein unit cell comprises at least one axis of rotational symmetry;
wherein said plurality of oligomeric protein unit cells interact with each other at one or more symmetrically repeated protein-protein interfaces to form said 2D protein array;
wherein said 2D protein array presents the protein of interest on its surface;
providing at least one potential binding target; and
determining if the at least one potential binding target is a binding partner of the protein of interest present on the surface of the 2D protein array.

31. The method of claim 30, wherein said determining comprises fluorescence resonance energy transfer (FRET).

32. The method of claim 31, wherein said protein of interest is labeled with a first detectable label, and wherein said at least one potential binding target is labeled with a second detectable label.

33. The method of claim 32, wherein said first detectable label comprises a first fluorescent label and said second detectable label comprises a second fluorescent label.
